(19) United States
(12) Patent Application Publication
(10) Pub. No.: US 2014/0081154 A1
(43) Pub. Date: Mar. 20, 2014

(54) DEVICES, SYSTEMS, AND METHODS FOR ASSESSING IMPLANTS, ORGANS, TRANSPLANTS, TISSUES, SYNTHETIC CONSTRUCTS, VASCULAR GRAFTS, AND THE LIKE

(71) Applicant: Landy Toth, Newtown, PA (US)

(72) Inventor: Landy Toth, Newtown, PA (US)

(21) Appl. No.: 14/118,366

(22) PCT Filed: May 16, 2012
(86) PCT No.: PCT/US12/38052

§ 371 Date: Nov. 18, 2013

Related U.S. Application Data
(60) Provisional application No. 61/486,992, filed on May 17, 2011.

(51) Int. Cl. A61B 5/00; A61M 1/10; A61F 2/24; A61B 5/026; A61F 2/06

(52) U.S. Cl. CPC A61B 5/4851; A61B 5/0261; A61B 5/686; A61B 5/0002; A61F 2/06; A61F 2/2472; A61M 1/10
USPC 600/479; 600/476; 600/300; 600/504

(57) ABSTRACT

A system for monitoring a body includes a surgical implant configured for implantation within a body, a sensory module coupled to the surgical implant and configured for implantation into the body in conjunction with the surgical implant, and a communication module coupled to the surgical implant and configured for implantation into a body in conjunction with the surgical implant. The sensory module is configured to monitor characteristics of the surgical implant, surrounding tissue and/or adjacent tissue. The communication module is electrically coupled to the sensory module and is configured to communicate a signal derived from said characteristics to an external entity.

### CROSS-REFERENCE TO RELATED APPLICATIONS

This application claims the benefit of, and priority to, U.S. Provisional Patent Application No. 61/486,992, filed on May 17, 2011, the entire contents of which are hereby incorporated by reference herein.

### TECHNICAL FIELD

The present disclosure is directed to devices, systems, and methods for monitoring tissue, an organ, an implant, and/or a transplant within a body. More particularly, the present disclosure is directed to devices, systems, and methods for monitoring the operation of an organ or health of a tissue segment within a body; monitoring the interface between an implant and the tissues and/or organs adjacent to or surrounding the implant; monitoring an artificial or synthetic transplant within a body; integrating diagnostic functionality to a synthetic or tissue engineered implant or transplant; interfacing electromechanical elements with an organ; and determining the patency of a vascular graft or stent.

### BACKGROUND

Over 46 million inpatient surgical procedures were performed in the United States in 2009. Many of these surgical procedures involved internal surgery on an organ, transplantation of an organ, and/or the implantation of a medical device. Needless to say, during the post-surgical recovery period patient health, care, and quick recovery are paramount to the successful outcome of such procedures.

At the same time, there is also a need to lower healthcare expenditures while simultaneously improving patient outcomes and recovery times in the post-surgical setting.

One critical aspect of ensuring a quick and reliable recovery is to uncover post-surgical complications early so that they can be acted upon before an emergency situation arises. Early detection provides medical staff with the time needed to perform less invasive interventions and to attempt more cost effective therapies to improve the patient outcome. Early prediction of such complications often requires assessment of the surgical site, associated implants and/or transplants, surrounding tissues and/or organs, and the like.

Coronary artery disease affects approximately seven million Americans, causing 1.5 M myocardial infarctions and over half a million deaths per year at an estimated cost exceeding $100B. Over 1 million percutaneous coronary interventions (PCI) and over 350,000 coronary artery bypass surgeries (CABG) are performed in the US annually. During a CABG procedure, arteries or veins are grafted to the coronary arteries to bypass atherosclerotic narrowing and improve blood supply to the myocardium. During a PCI procedure one or more stents may be applied to relieve blockages and improve blood flow. A graft or stent is considered patent so long as there is flow through the graft or stent without significant stenosis (>70% diameter of the graft or stent). Graft patency is dependent on several factors including type (internal thoracic artery, radial artery, or great saphenous vein), the size of the artery to which the graft is anastomosed, handling of the graft during the procedure, and the skill of the surgeon performing the procedure.

In general, vein grafts have worse patency rates than those formed with internal thoracic arteries and radial arteries. To compensate, a sleeve may be placed around the vein graft to reinforce the graft and dramatically improve patency.

Yet there remains a need to determine the patency of a vascular graft or stent in an efficient and cost effective manner. There is a need to monitor and predict future complications that may arise within a graft or stent. In addition, there remains a need to determine blood flow through a vascular graft or stent.

There is a need to determine the patency of implanted stents and grafts used in angioplasty, coronary bypass, carotid bypass, peripheral bypass, dialysis grafts, and other procedures, as well as for cerebro-spinal fluid shunts and other shunts.

There is also a need to efficiently and cost-effectively determine organ function after a surgery and/or in high risk persons.

There is a need to provide long-term health monitoring of patients after a surgery and/or high risk patients in a minimally invasive, efficient, and cost effective manner.

This is also a need to closely and efficiently monitor surgical sites and associated organs during and after the surgical procedure until the patient has fully recovered.

### SUMMARY

One objective of the present disclosure is to provide a system and method for monitoring a body and, more particularly, an internal surgical site, tissue adjacent to or surrounding the surgical site, and/or an organ inside a body.

A further objective is to provide a system and method for early and predictive detection of postsurgical complications particularly relevant to the recovery and long-term outcome of the surgical site, surrounding and adjacent tissues, and organs associated with the surgical site.

Yet another objective is to provide a system and method for evaluating function and performance of an implant or transplant within a body.

Another objective is to provide a system and method for continuously monitoring the patency of a vascular graft.

Yet another objective is to provide a self-diagnostic vascular graft.

Another objective is to provide a self-diagnostic synthetic biomaterial construct.

Another objective is to provide a self-diagnostic transplanted or synthetic organ, tissue, or graft.

Yet another objective is to provide a system and method for improving the patency of a vascular graft.

Another objective is to provide a system and method for long-term monitoring of flow through a lumen in a body. A very particular objective is to provide a system and method for long-term monitoring of flow through a vascular graft.

Another objective is to provide a self-diagnostic system for enhancing blood flow to the myocardium.

Another objective is to provide a system for monitoring flow loss parameters over a length of a lumen in the body.

Yet another objective is to provide a system for non-contact monitoring of a vascular graft.

The above objectives are wholly or partially met by devices, systems, and methods described herein. In particular, features and aspects of the present disclosure are set forth in the appended claims, following description, and the annexed drawings.

In accordance with aspects of the present disclosure, a system for monitoring a body is provided including a surgical implant configured for implantation into a body, a sensory module coupled to the surgical implant and configured for implantation into the body in conjunction with the surgical implant, and a communication module coupled to the surgical implant and configured for implantation into the body in conjunction with the surgical implant. The sensory module is configured to monitor characteristics, e.g., physiological and/or anatomical characteristics, of the surgical implant, surrounding tissue and/or adjacent tissue. The communication module is electrically coupled to the sensory module and is configured to communicate a signal derived from said characteristics to an external entity.

In aspects, the surgical implant includes a complaint scaffold. In such aspects, the sensory module and/or the communication module may be affixed to the complaint scaffold. Further, the compliant scaffold may itself be the surgical implant, or the compliant scaffold may be configured to provide intimate contact with the surgical implant.

In aspects, the surgical implant is a vascular graft and the compliant scaffold is configured for positioning about the vascular graft. The compliant scaffold may alternatively or additionally be configured to fit to a mesh, a general graft, or an organ surface.

In aspects, the communication module is electrically connected to the compliant scaffold and at least a portion of the compliant scaffold provides an antenna function configured to facilitate communication with the external entity.

In aspects, the compliant scaffold includes at least one electrically conductive region electrically connected to the communication module and/or the sensory module. The at least one electrically conductive region is configured to electrically interface with the surgical implant.

In aspects, the sensory module and/or the communication module includes one or more eyelets configured to facilitate attachment of the sensory module and/or the communication module to the surgical implant.

In aspects, a power supply is provided. The power supply is disposed in electrical communication with the sensory module and/or the communication module and may be affixed to the compliant scaffold.

In aspects, the sensory module, the communication module, and/or the power supply are electrically connected by at least one flexible link. The at least one flexible link may be formed from a stretchable interconnect including at least one electrically insulating region and at least one electrically conducting region.

The electrically insulating regions may be formed from one or more polymers selected from the group consisting of poly(dimethylsiloxane), perfluoropolyether, silicone-containing polyurethane, polyurethane, PFPE-PDMS block copolymers, polyisoprene, polybutadiene, fluoroolefin-based fluoroelastomers. The electrically conducting regions may be formed from one or more conducting materials selected from the group consisting of poly(3,4-ethylenedioxythiophene), polyaniline, gold, silver, carbon, copper, tin, platinum, nickel, titanium, chromium, aluminum, and alloys thereof.

In aspects, the sensory module, the communication module, and/or the power supply are comprised of physically distinct components distributed over the surgical implant. The physically distinct components may include passive elements, silicon chips, ASICs, sensors, actuators, RF components, signal conditioning components, and mixed signal silicon dies. The system may further include one or more flexible links adapted so as to electrically interconnect the physically distinct components.

In aspects, the sensory module is configured to monitor motion, e.g., through use of an accelerometer, gyroscope, spring coil, resonant vibrating element, vibration sensitive switch, or the like to convey movement information.

In aspects, the sensory module includes at least one light source configured to illuminate the surgical implant, surrounding tissue and/or adjacent tissue, and at least one photodetector configured to receive light from the surgical implant, surrounding tissue and/or adjacent tissue.

In aspects, the communication module and/or the power supply may be configured to harvest energy, e.g., RF energy, from an external energy source.

The system may further include a stimulation module, e.g., stimulation electrodes, in electrical communication with the communication module and configured to stimulate the surgical implant and/or tissue associated with the surgical site.

In aspects, a plurality of sensory modules may be provided. Each sensory module is configured to monitor physiological and/or anatomical characteristics at a different location along the surgical implant, surrounding tissue and/or adjacent tissue.

According to aspects, there is provided a system and method for early and predictive detection of postsurgical complications particularly relevant to the recovery and long-term outcome of a surgical site, surrounding tissues, organs and/or transplants within a body. The system includes a sensory module adapted to read information, e.g., physiological and/or anatomical information, from a surgical site, associated tissues, organs, implants, and/or transplants and communicate a related signal to a communication module. The communication module is arranged so as to exchange information relating to the signal, system information, and/or system health with an external device, network, or person located outside of the body.

According to aspects, there is provided a system and method for evaluating function and performance of a transplant within a body. The system includes a sensory module and a communication module in electrical communication with each other. The sensory module is adapted to generate a signal related to the transplant e.g., physiological and/or anatomical characteristics thereof, and communicate a related signal to the communication module. The communication module is arranged so as to exchange information relating to the signal, system information, and/or system health with an external device, network, or person located outside of the body.

Other objectives include providing a system and method for continuously monitoring the patency of a vascular graft, providing a self-diagnostic vascular graft (SDVG), and providing a system for monitoring patency of a vascular graft.

In accordance with aspects of the present disclosure and the above-identified as well as other objectives, a system for monitoring patency of a vascular graft is provided. They system includes a compliant scaffold formed about a vascular graft, a sensory module affixed to the compliant scaffold and configured to monitor characteristics, e.g., physiological and/or anatomical characteristics, of the vascular graft, surrounding tissue and/or adjacent tissue, a communication module affixed to the complaint scaffold and electrically coupled to the sensory module, and an antenna affixed to the compliant scaffold and electrically coupled to the communication module.

In aspects, the antenna is formed from flexible conducting material configured to conform to a surface of the compliant scaffold.

In aspects, the antenna is interwoven into the compliant scaffold.

In aspects, the compliant scaffold is at least partially formed from an electrically conducting material and the antenna is formed from at least a portion of the compliant scaffold.

In aspects, the sensory module is configured to monitor blood flow through the vascular graft. A plurality of sensory modules may be provided to monitor blood flow at various different positions along the vascular graft, surrounding tissue and/or adjacent tissue.

In aspects, sensory module includes at least one light source directed towards the vascular graft and at least one photodiode and/or photodetector directed towards the vascular graft.

In aspects, the sensory module includes at least one electrode configured to interface with the vascular graft.

In aspects, one or more flexible links electrically couple the sensory module and the communication module to one another.

In aspects, the system further includes a power supply affixed to the compliant scaffold and configured to power the sensory module and/or the communication module.

The system may be further configured similarly to any of the other aspects described herein.

Another objective is to provide a self-diagnostic synthetic biomaterial construct comprising a tissue engineered construct formulated so as to mimic the physical properties and shape of at least a portion of an organ, or other tissue structure. In particular, provided is a self-diagnostic system including a tissue engineered construct configured for compatibility with body tissue, and a sensory module at least partially embedded into the tissue engineered construct. The sensory module is configured to monitor characteristics, e.g., physiological and/or anatomical characteristics, of the tissue engineered construct, surrounding tissue and/or adjacent tissue.

In aspects, a communication module is at least partially embedded into the tissue engineered construct and is electrically coupled to the sensory module. The communication module is configured to communicate a signal derived from said characteristics to an external entity.

In aspects, the tissue engineered construct is fabricated so as to mimic a body vessel, e.g., a vascular graft.

In aspects, the sensory module is configured to monitor blood flow through the tissue engineered construct.

In aspects, the tissue engineered construct is fabricated so as to mimic at least a portion of a heart.

In aspects, the system further includes a power supply at least partially embedded into the tissue engineered construct.

Yet another objective is to provide a system for remote monitoring of a vascular graft. The system includes a ringlet housing configured to surround a portion of a vascular graft, at least one electrode, e.g., an electrode set, disposed within the ringlet housing, and a communication module electrically coupled to the at least one electrode. The communication module is configured to energize the at least one electrode so as to generate an electromagnetic field within the vascular graft. The communication module is further configured to monitor a current within the at least one electrode.

In aspects, the ringlet housing and at least a portion of the at least one electrode are formed from a stretchable interconnect comprising one or more electrically insulating regions and one or more electrically conducting regions.

In aspects, the at least one electrode is further configured to function as an antenna and the communication module is configured to interface with the antenna function of the at least one electrode to communicate with an external entity.

In aspects, the ringlet housing further includes at least one attachment point configured to facilitate affixing the ringlet housing to the graft or adjacent tissue.

In aspects, the system further includes a power supply disposed within the ringlet housing and configured to power at least one of the electrode(s) and the communication module.

In aspects, the at least one electrode includes an EM electrode set.

Another objective is to provide a method for determining the patency of a vascular graft including attaching a compliant scaffold in accordance with any of the aspects described herein to a vascular graft, implanting the vascular graft into a body, monitoring characteristics, e.g., physiological and/or anatomical characteristics, and communicating data related to the characteristics to an external entity, in accordance with any of the aspects described herein.

Yet another objective is to provide a method for fabricating a self-sensing tissue engineered construct including growing a tissue engineered construct, embedding a sensor module in accordance with any of the aspects described herein into the tissue engineered construct, monitoring characteristics, e.g., physiological and/or anatomical characteristics, and communicating data related to the characteristics to an external entity, in accordance with any of the aspects described herein.

These methods may further include coating the sensor modules with a biocompatible coating.

### BRIEF DESCRIPTION OF THE DRAWINGS

FIG. 1—Shows a schematic of a system for monitoring a site in a body in accordance with the present disclosure.

FIG. 2—Shows a self-diagnostic vascular graft in accordance with the present disclosure.

FIG. 3—Shows another self-diagnostic vascular graft in accordance with the present disclosure.

FIG. 4—Shows another self-diagnostic vascular graft in accordance with the present disclosure.

FIG. 5—Shows a close up of a system for monitoring the patency of a vascular graft in accordance with the present disclosure.

FIGS. 6a, 6b, and 6c—Show non-limiting examples of flexible links configured for coupling a remotely attached sensory module and a communication module to one another in accordance with the present disclosure.

FIG. 7—Shows an electro-optic sensory module positioned within a system in accordance with the present disclosure.

FIG. 8—Shows exemplary waveforms obtained from an electro-optical sensory module related to blood flow through at adjacent tissue site in accordance with the present disclosure.

FIGS. 9a and 9b—Show non-limiting examples of sensory modules for attachment to a compliant scaffold in accordance with the present disclosure.

FIG. 10—Shows an antenna woven into a compliant scaffold in accordance with the present disclosure.

FIG. 11—Shows a strategically woven compliant scaffold including multiple electrically addressable regions in accordance with the present disclosure.

FIG. 12—Shows another strategically woven compliant scaffold including multiple electrically addressable regions in accordance with the present disclosure.

FIG. 13—Shows a ringlet sensor for monitoring patency of a vascular graft in accordance with the present disclosure.

FIG. 14—Shows a non-limiting example of a ringlet sensor for monitoring patency of a vascular graft in accordance with the present disclosure.

FIG. 15—Shows another ringlet sensor for monitoring patency of a vascular graft in accordance with the present disclosure.

FIG. 16—Shows a non-limiting example of an electrically shielded system for monitoring a vascular graft.

FIGS. 17a to 17d—Show a system for monitoring a site within a body in accordance with the present disclosure.

FIG. 18—Shows a multi-component system for monitoring function of an organ in accordance with the present disclosure.

FIGS. 19a and 19b—Show non-limiting examples of multi-component systems for monitoring one or more bodily functions of a subject in accordance with the present disclosure.

### DETAILED DESCRIPTION OF THE DRAWINGS

To the extent they are consistent with one another, any of the aspects described herein may be used in conjunction with any or all of the other aspects described herein. Further, the term tissue as used herein refers broadly to any organ, vessel, or other anatomical structure within or forming part of the body.

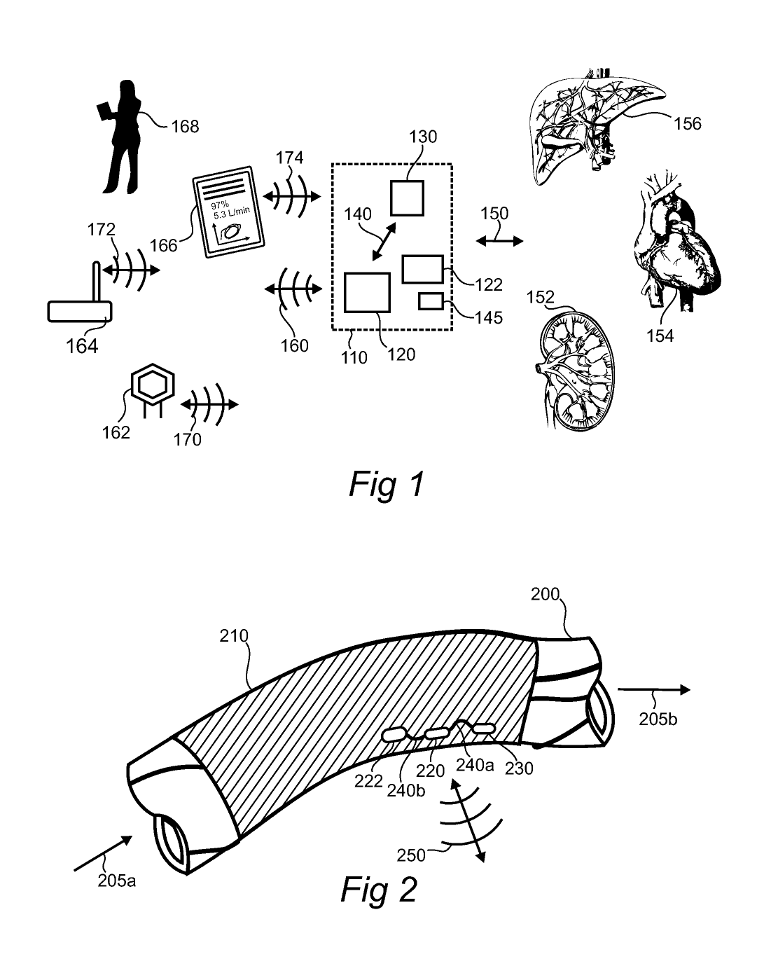

FIG. 1 shows a schematic of a system 110 for monitoring a surgical site in a body. In aspects, the system 110 includes a sensory module 130, a communication module 120, and a power supply 122. The sensory module 130 may be configured to provide an optionally bidirectional interaction signal 150 to measure physiological and/or anatomical information about tissue including organs, e.g., kidney 152, heart 154, and/or liver 156, or other body tissue, a surgical site (including tissue surrounding and adjacent to the surgical site), implant, transplant, lumen wall, or the like. The sensory module 130 communicates one or more signals 140 with the communication module 120 related to the interaction signal 150. The communication module 120 interacts with the sensory module 130 and an external entity such as a reader/repeater 162, a network hub 164, a mobile device 166, a person 168, or the like, e.g., via signals 160, 170, 172, 174. The information conveyed by the communication module 120 may be related to the interaction signal 150, may include an alarm, alert, diagnostic signal, combination thereof, or any other signal related to the state of the surgical site, tissue, organ, implant, lumen, transplant, etc. under observation.

The system 110 may include a plurality of sensory modules 130, each configured for one or more similar or different functions in order to elucidate further and/or redundant information about the organ, surgical site, implant, transplant, lumen wall, tissue, or the like. One or more sensory modules 130 may be arranged to measure or monitor such physiological parameters as analyte concentrations, partial pressures of gaseous species, flow of a fluid, turbidity, electromagnetic absorption, pressure, pressure gradients, electromagnetic reflectance, acoustic impedance, electrophysiological activity, electrical impedance, temperature, temperature gradients, mechanical impedance, pressure, accelerations, and the like. One or more sensory modules 130 may also be configured to monitor the concentration of a chemical species such as for example, glucose levels, pH, sugar, blood oxygen, glucose, moisture, radiation levels, chemical activity, ionic species, enzymatic species, oxygen, carbon dioxide, and the like.

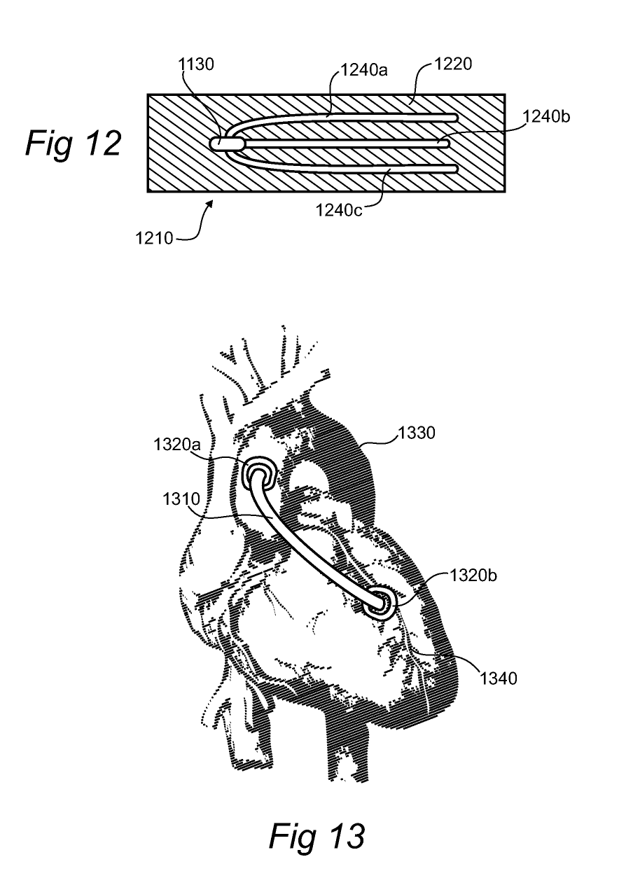

The system 110 may include one or more electrical pacing leads, e.g., in the configuration of electrically addressable regions 1140, 1150, 1160 (FIG. 11), electrically addressable regions 1240a, 1240b, 1240c (FIG. 12), or sensory modules 1640a-f (FIG. 16), so as to stimulate local tissues, perhaps in response to a monitored physiological parameter, etc.

In aspects, the sensory module 130 may be configured to monitor at least a portion of the visible absorption spectrum of the tissues surrounding or adjacent to a surgical site or as part of an associated organ, transplant, etc. In such aspects, the sensory module 130 may include one or more light sources, such as narrow bandwidth light emitting diodes, broad bandwidth light emitting sources, or other suitable light sources. In addition, the sensory module 130 may include ultraviolet or near or far infrared emitting sources. In addition, the sensory module 130 may include one or more photodetectors, photodiodes, phototransistors, PIN photodiodes, or the like arranged to detect incident light either emitted from an associated light source (e.g., a diode, a light emitting diode, a diode laser, a fiber optic element, etc.), from a light source externally located from the body, an ambient light source, or from a fluorescent source located within the sensory module 130 or the surrounding and/or adjacent tissues.

The sensory module 130 may include a pulse-echo ultrasound subsystem, so as to monitor local tissue density, changes in local tissue thickness, to determine internal structures of an adjacent organ, tissues, graft, or the like.

In aspects wherein the sensory module 130 may be configured with multiple light sources and photodetectors, e.g., in the configuration of electrically addressable regions 1140, 1150, 1160 (FIG. 11), electrically addressable regions 1240a, 1240b, 1240c (FIG. 12), or sensory modules 1640a-f (FIG. 16), the system 110 may be configured to form a map of absorptive and/or reflective properties of tissues, organs, implant, transplant and the like around the surgical site. In such aspects, the map may be used to monitor local changes in tissue absorption as a means to detect local variation in analyte concentration, local tissue damage, etc. that may be missed by any single sensor.

In aspects, the sensory module 130 may be configured to monitor the bioimpedance and/or collectively the impedance tomography of tissues adjacent thereto. In such aspects, the sensory module 130 may include one or more electrodes, e.g., in the configuration of electrically addressable regions 1140, 1150, 1160 (FIG. 11) or electrically addressable regions 1240a, 1240b, 1240c (FIG. 12), fashioned so as to conductively or capacitively interact with adjacent tissues. Any pair or plurality of electrodes may be excited or probed in order to monitor the impedance or construct a map of impedance around the surgical site, associated organ(s), tissue, implant or the like.

In aspects, the sensory module 130 may be configured to monitor local motion and motion artifacts in and around the surgical site. In such aspects, the sensory module 130 may contain an accelerometer, gyroscope, spring coil, resonant vibrating element, vibration sensitive switch, or the like to convey movement information. The information can be used to eliminate motion artifacts from other sensors in one or more sensory modules 130 as well as to provide feedback related to trauma, synchronization of readings with local movement, monitoring of flow related movements (e.g., pulsatile flow related to normal or abnormal blood flow), wear at an implant surface, relative movement of objects near the surgical site, and the like.

In aspects, the implants and/or associated organs or tissues may be subjected to a dynamic environment, under continual undulation, etc. In such aspects, the system 110 may include one or more features configured to improve monitoring capability in such environments by compensating for movement artifacts (e.g., by synchronizing physiological measurements with movement, by removing movement signals from physiological readings, etc.) and/or providing means for in-growth of the system 110 into the surrounding and adjacent tissues, thus minimizing relative movement between one or more of the sensors included in the system 110 and the organ/tissue with which the system 110 interfaces.

In aspects, the sensory module 130 may be configured to monitor local strain. In such aspects, the sensory module 130 may contain a soft elastomeric strain gauge, a piezoresistive strain gauge, a capacitive strain gauge, or the like. In particular, a capacitive elastomeric strain gauge can be used to determine large strains in soft tissues in and around the surgical site.

In aspects, the sensory module 130, communication module 120, and/or power supply 122 can be physically combined to form a single unit. In addition, some functional aspects of the sensory module 130, communication module 120, and power supply may be interchanged without substantially altering the overall operation of system 110.

The communication module 120 may include RF circuitry including an antenna, matching network, amplifiers, and the like to suitably communicate with an entity outside of the body, e.g., a reader/repeater 162, a network hub 164, a mobile device 166, a person 168, or the like. The communication module 120 may additionally or alternatively include an RF transceiver, a transponder, or a transmitter to communicate with the outside entity.

Alternatively or in combination, the communication module 120 may be configured for optical communication, ultrasonic communication, acoustic communication, and/or conductive communication. In general, any suitable communication medium may be used to communicate between the communication module 120 and an outside entity.

The system 110 may include a power source 122 and associated circuitry. In such aspects, the power source 122 may be a primary or rechargeable battery, a thin film battery, an energy harvesting system, a nuclear power source, a fuel cell source, an electrochemical source, a bio-electrochemical source, or the like. In the case of a rechargeable power source, the power source may be recharged by an externally applied RF signal so as to extend the functional life of the implant.

Alternatively, additionally, or in combination, the system 110 may derive power from an external source such as an RF source. In such aspects, the power source 122 may further include associated circuitry to collect and store sufficient incoming power to power the communication module 120, the sensory module 130, and any other components of the system 110.

The system 110 may include a super capacitor 145 configured to receive energy from an associated energy harvesting module, RF source, or the like. The super capacitor 145 may be configured to accumulate energy from the associated source and to provide high current pulses for operation of the one or more aspects of the system 110 during use. In one non-limiting example, the system 110 may include a super capacitor 145 and a regulator (incorporated into super capacitor 145 or one of the other components of system 110), such that a sufficiently high current pulse with stable and predictable parameters may be delivered to one or more components (e.g., electrode, sensor, optical component, etc.) during operation. The super capacitor 145 and/or associated regulator may be included in power supply 122, the communication module 120, the sensory module 130, other component of the system 110, or may be a separate component.

In aspects, the system 110 may be configured to monitor one or more functions of surrounding or adjacent tissue (including organs, e.g., blood flow into and/or out of the organ), local tissue health, local neural activity, changes in local tissue density, local odema formation, etc. The system 110 may be configured to monitor an implant, surrounding tissue, and/or adjacent tissue to monitor progression of tissue/implant contact, scar formation, implant viability, tissue ingress into the implant, etc. In further aspects, the system 110 may be configured to monitor an associated artificial and/or synthetic implant surrounding tissue, and/or adjacent tissue within a subject via one or more approaches in accordance with the present disclosure and/or to provide an interface between body function and the system 110 (e.g., a cybernetic function, neural interface, etc.).

In aspects, the system 110 may be integrated into a body modification implant (e.g., a subdermal implant, a stud, a horn, a ring, etc.). In such aspects, the body modification implant and integrated hardware may be implanted just under the surface of the body, thus creating a fashionable body modification (e.g., a functional, yet fashionable implanted system). The body modification implant may communicate with an external entity by one or more methods (e.g., introduction of lights, acoustic elements, pressure contacts, etc.), each of which may be included into the associated system 110 under the skin. The body modification implant may have a small power source, or may obtain adequate power by an energy harvesting method, and/or may also be recharged or powered by an external source.

In aspects, the system 110 may be integrated into an implant so as to improve functionality thereof, add diagnostic capabilities thereto, catch early complications that may occur after implantation, etc. The system 110 may be used to determine functionality and/or seating of the implant against the local tissues, allow for coupled scanning of the implant with external imaging systems, or the like.

In aspects, the system 110 may be integrated into a catheter element, and/or integrated into a venous graft so as to interact with an associated catheter element placed therein. In one non-limiting example, the system 110 may include a sensory module 130 configured to detect the presence of an associated catheter element located within the graft. According to such aspects, the system 110 may be used to determine when and/or assist with accurate placement of the catheter element within the graft. Such information may be advantageous for positioning surgical tools within the graft, for determining adequate placement of a catheter element within a venous graft (e.g., for safe dialysis, etc.).

In aspects, the system 110 may include a cord-like or worm-like feature (e.g., a string, a wire, etc.) extending from the portion of the system 110 located at the surgical site, to an access point located on the body of the subject. Such a feature may be advantageous for easy removal of the system 110 from the subject after the monitoring period has been completed.

FIG. 2 shows a self-diagnostic vascular graft (SDVG) provided in accordance with the present disclosure. The SDVG includes a vascular graft 200 formed from an internal thoracic artery, radial artery, great saphenous vein, tissue engineered vessel, synthetic vascular graft, engineered graft, stent, stent graft, polymeric graft, or the like. As shown in FIG. 2, a compliant scaffold 210 may be placed around the vascular graft 200. The compliant scaffold 210 may be secured to the vascular graft 200 using any suitable method known in the art. The compliant scaffold 210 may further be coated with a biocompatible coating and/or a bioadhesive to optimize the connection between the scaffold 210 and the vascular graft 200. The SDVG further includes a sensory module 230 affixed to or interwoven into the compliant scaffold 210. The sensory module 230 may be electrically connected to a communication module 220. The communication module 220 may likewise be affixed to or interwoven into the compliant scaffold 210. A power supply 222 is likewise affixed or interwoven into the compliant scaffold 210 and is electrically coupled to the communication module 220 and/or the sensory module 230. The sensory module 230, the communication module 220, and/or the power supply 222 may be interconnected by one or more links 240a, 240b. The communication module 220 may include an integrated antenna for communicating with an external entity to the body, e.g., as indicated by bidirectional signal 250.

In general, the sensory module 230, the communication module 220, the power supply 222, and the one or more links 240a, 240b may be adapted so as not to impede the compliance, openness, or profile of the compliant scaffold 210 over the vascular graft 200. Furthermore, the sensory module 230, communication module 220, power supply 222, and the one or more links 240a, 240b may be sufficiently small and unobtrusive so as to minimally impact pressures applied to the vascular graft 200 after attachment of the compliant scaffold 210. They may additionally be of sufficiently low profile so as to minimize dynamical stresses and abrasive forces caused by relative motion between the vascular graft 200 and adjacent tissues. In aspects, the sensory module 230, communication module 220, power supply 222, and one or more links 240a, 240b may be coated with a lubricious, biocompatible coating so as to further minimize any of the above adverse effects. In aspects, the sensory module 230 may have a characteristic length of less than 1 mm, less than 0.5 mm, or less than 0.25 mm. In aspects, the sensory module 230 and/or communication module 220 may have a characteristic thickness of less than 1 mm, less than 0.5 mm, or less than 0.2 mm.

In aspects wherein an electrically conducting compliant scaffold 210 may be provided, the communication module 220 may be electrically connected to the compliant scaffold 210 to facilitate various functions. In aspects, the electrical connection between the compliant scaffold 210 and communication module 220 may perform the function of the link 240a, e.g., facilitating communication between the communication module 220 and the sensory module 230.

In aspects, and particularly for providing enhanced profile and simplicity, the electrical connection between the compliant scaffold 210 and communication module 220 may be used to connect RF circuitry within the communication module 220 to the compliant scaffolding 210. In such aspects, the scaffolding 210 facilitates at least a portion of the role of an antenna to facilitate efficient communication between the communication module 220 and an external entity to the body. In this aspect, the carrier frequency, and dimensions of the scaffolding 210, RF circuitry in the communication module 220, and the surrounding tissues and the location of the graft 200 in the body all factor into the interaction and efficiency of the compliant scaffolding 210 functioning as an antenna.

The links 240a, 240b generally include one or more conducting elements that facilitate power and/or data flow between the sensory module 230, the communication module 220, and the power supply 222. The links 240a, 240b may be formed from a flex circuit, a multi-wire bundle, a braided wire bundle, a stretchable interconnect, or the like.

In aspects, the SDVG, complete with vascular graft 200, may be constructed so as to provide a radial mechanical compliance similar to that of an internal thoracic artery.

In aspects, the compliant scaffold 210 may be at least partially formed from an electrically conducting wire such as a metal (e.g., gold, platinum, etc.), transition metal (e.g., tantalum), metal alloy (e.g., stainless steel, a cobalt alloy, Co—Cr—Ni—Mb, etc.), and/or a shape memory alloy. One exemplary shape memory alloy is a nickel titanium alloy often referred to as nitinol. Other non-limiting examples of shape memory alloys that may be used include Cu—Al—Ni, Pt alloys, Co—Ni—Al, Ti—Pd, Ni—Ti, and the like. Alternatively, the compliant scaffold 210 may be formed from a composite or laminate of insulating material such as a polymer (e.g., a silicone, a polyethylene, a polyurethane, a bio absorbable polymer, etc.), and a conducting material such as a metal, metal-composite, carbon, or a conjugated polymer. A conjugated polymer coating may provide a suitable biocompatible interface as well as facilitate at least a partial role as a conductor for RF communication purposes. The compliant scaffold 210 may include one or biodegradable polymers such as collagen, polyesters, polyorthoesters, polyanhydrides, resorbable polymers, combinations thereof, and the like.

The sensory module 230, communication module 220, and power supply 222 may be affixed to the compliant scaffold 210 using an adhesive, by welding, brazing, soldering, by suturing, tying, or the like. Alternatively, the sensory module 230, the communication module 220, and/or the power supply 222 may include a mechanically interlocking element that allows for simple fixation of the associated module 220, 230, 222 to the compliant scaffold 210.

The sensory module 230 may include any of the options, features, or configurations discussed above with respect to sensory module 130 (see FIG. 1). In particular, the sensory module 230 may include one or more light sources and one or more photodiodes, photodetectors, phototransistors, or the like. In general, the light source may be arranged to face into the vascular graft 200. One or more sensory modules 230 may be arranged around the circumference of the vascular graft 200. Light from a single module 230 may be received by multiple modules 230 to piece together absorptive qualities of the vascular graft 210 and the blood flowing through it (as indicated by arrows 205a, 205b). The sensory module 230 may further include chemiluminescent indicators indicative of an analyte such as oxygen, carbon dioxide, glucose, and the like. In this case, changes in the analyte may be monitored so as to determine the overall health and/or tone of the tissue local to that sensory module 230. Degradation of tissue tone, a reduction in oxygen concentration, or an increase in carbon dioxide concentration in the graft tissues and/or the blood may all be indicative of an obstruction or degradation of the vascular graft 200.

The power supply 222 may likewise include any of the options, features, or configurations discussed above with respect to power supply 122 (see FIG. 1).

In aspects, tissue engineered constructs and vessels may be constructed and precursor materials selected as is known in the prior art. For example, International Patent Application Nos. PCT/US2010/49850, PCT/US2010/47725, PCT/US2009/59547, PCT/US2010/50460, PCT/US2010/39165, PCT/US2009/46407, PCT/US2010/34662, and PCT/US2010/32234, PCT/US2010/29952, and US Patent Application Publication Nos. 2010/0752708 and 2009/0457507 contain a range of precursor materials and methods for fabricating tissue engineered constructs that may be suitable for use herein and are incorporated herein by reference in their entirety.

In aspects, a system in accordance with the present disclosure may be integrated into one or more tissue engineered constructs, vessels, sheets, and/or organs by one or more methods. A method for integrating a foreign sensory body (e.g. a system, a sensory module, a communication module, etc.) into a tissue engineered construct (e.g. a synthetic organ) during a fabrication process includes introducing the hardware or foreign body into the construct as it is being grown. The foreign body may be seeded with a coating of biocompatible seed molecules so as to ensure that the foreign body may be tightly integrated into the construct. The coating may dramatically reduce foreign body response and rejection of the hardware into the tissue construct.

The above described devices, systems, and methods may similarly be used for monitoring patency of a stent. With regard to stents, the aspects detailed above apply similarly except that they would be placed within a vessel in the body. Thus, instead of securing the sensory module 230, communication module 220, and/or power supply 222 to the compliant scaffold 210, these components would be secured to the stent in regions that do not undergo significant deformation during an expansion procedure.

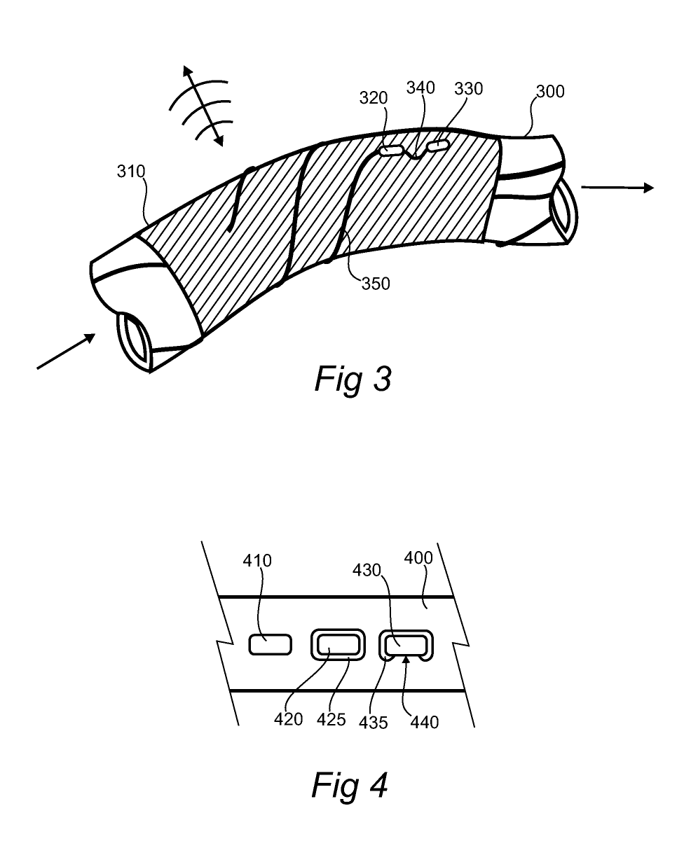

FIG. 3 shows another self-diagnostic vascular graft (SDVG) provided in accordance with the present disclosure. As shown in FIG. 3, the SDVG includes a vascular graft 300 and optionally a compliant scaffold 310 attached to the vascular graft 300. The SDVG further includes a sensory module 330 affixed to the compliant scaffold 310 and a communication module 320 also affixed to the scaffold 310. The sensory module 330 and the communication module 320 may be electrically connected via one or more links 340. Furthermore, the SDVG includes an antenna 350, e.g., a relatively soft, flexible, compliant antenna that is electrically connected to the communication module 320 in close proximity to, e.g., defining a low profile with respect to, the compliant scaffold 310.

In aspects, the compliant antenna 350 may be interwoven into the compliant scaffold 310. Alternatively, the antenna 350 may be strategically affixed to the scaffold 310 at one or more points along its length so as to ensure that the antenna 250 remains in a low profile configuration during use. In general, the compliant antenna 350 may be arranged so as not to significantly impede the compliance, openness, or profile of the compliant scaffold 310. The antenna 350 may be formed from an insulated wire, braided wire, a flex laminate, a microcoil, or the like. The antenna 350 may be strategically wound around the compliant scaffold 310 so as to form a helix. Alternatively, the antenna 350 may be formed into a loop extended over the surface of the compliant scaffold 310, or may be disposed about the scaffold 310 in any other suitable configuration. A power supply similar to those described above with respect to FIGS. 1 and 2 may also be provided and may be incorporated into or affixed to the complaint scaffold 310 similarly as the sensory module 330 and communication module 320 and may electrically communication with the sensory module 330 and/or communication module 320 via one or more links 340.

FIG. 4 shows a self-diagnostic synthetic construct (SDSC) provided in accordance with the present disclosure. The SDSC includes a tissue engineered construct 400. The SDSC also includes a sensory module 430, a communication module 420, and an antenna 410 all at least partially embedded into or otherwise secured to the tissue engineered construct 400. A power supply may also be provided similarly as the sensory module 430, communication module 420, and antenna 410. The sensory module 430, communication module 420, and antenna 410 may be collocated into a single unit or may be distributed throughout the tissue engineered construct 400 as physically distinct components. The sensory module 430 may generally be provided in electrical communication with the communication module 420 and the communication module 420 may be generally provided in electrical communication with the antenna 410, although the sensory module 430 and antenna 410 may also be configured to directly communicate with one another. Except in regions that require intimate electrical contact with the tissue engineered construct 400, such as around any optional electrode elements 440, the sensory module 430, communication module 420, and antenna 410 may be electrically isolated from the tissue engineered construct 400.

In aspects, securement of one or more of the sensory module 430, the communication module 420, the antenna 410, and/or combinations thereof to the tissue engineered construct 400 may be completed by use of micro structures (e.g. microneedles, microhooks, microsythes, etc.) constructed from one or more materials (e.g. metallic materials, polymers, semiconductors, biodegradable materials, drug-loaded polymers, composites, combinations thereof, etc.), with bioadhesives, sutures, embedded during fabrication thereof, and the like.

In aspects, similarly as described above, a plurality of sensory modules 430 may be provided. In such aspects, each sensory module 430 may be electrically connected to the communication module 420. In general, the sensory modules 430 may be distributed throughout the tissue engineered construct 400 in any suitable configuration.

The tissue engineered construct 400 may be fabricated using the methods and materials outlined herein. The tissue engineered construct 400 may be a sheet of tissue, a patch, an organ, or a graft. The tissue engineered construct 400 may be fabricated as a patch, tissue segment, portion or all of a bladder, abdominal mesh, lung, kidney, heart, pancreas, and the like.

The one or more sensory modules 430, communication module 420, and antenna 410 may be integrated into the tissue engineered construct 400 during the fabrication process thereof. In order to effectively integrate these components into the construct 400, they may be coated with a biocompatible coating 425, 435. Alternatively, they may be coated with a layer of seed cells suitable for forming strong coherent bonds with the tissue of the tissue engineered construct 400. In aspects, the sensory module(s) 430, communication module 420, and antenna 410 may be fully embedded into the tissue construct 400. This may help to improve acceptance of the construct 400 after implantation into a body.

In aspects, peptide amphiphiles may be suitable for use as a biocompatible coating to promote endothelialization and inhibit restenosis and thrombosis at the interface between the construct 400 and the surrounding tissues. Examples such as those described in International Patent Application No. PCT/US2009/63732, which is incorporated herein by reference in its entirety, may be suitable for such coatings.

In aspects, the tissue engineered construct 400 may be a tubule in the form of a synthetic vascular graft. In such aspects, the sensory module 430, communication module 420, and antenna 410 may be at least partially embedded into the wall of the vascular graft. The sensory module 430 may be arranged to monitor patency of the vascular graft, blood flow though the synthetic vascular graft, and/or monitor the walls of the graft for signs of rejection, changes in wall properties such as a change in density or thickness of the wall, an analyte concentration in the wall, oedema, material build-up inside the graft, or the like. The sensory module 430 may alternatively or additionally be configured to monitor the state of an anastomosis near the edge of a graft, and/or detect gap formation, wall thinning, wall thickening, and the like near the anastomosis. The sensory module 430 may further be configured to monitor surrounding and/or adjacent tissue. In this way, the sensory module 430 may be configured to monitor the patency of the graft in a variety of meaningful ways.

Alternatively or in addition, the sensory module 430 may be equipped to monitor an aspect of the electrophysiological function of the heart, thus providing the capability of providing more general health diagnostics for an indefinite term following the surgical implantation of the graft.

In aspects, the communication module 420 contains sufficient identification information so as to track the physical and anatomical properties of the graft as well as the history, serial ID, and the like of the graft.

In aspects, the one or more sensory modules 430 may be arranged so as to monitor the bioimpedance of the synthetic tissue engineered construct 400.

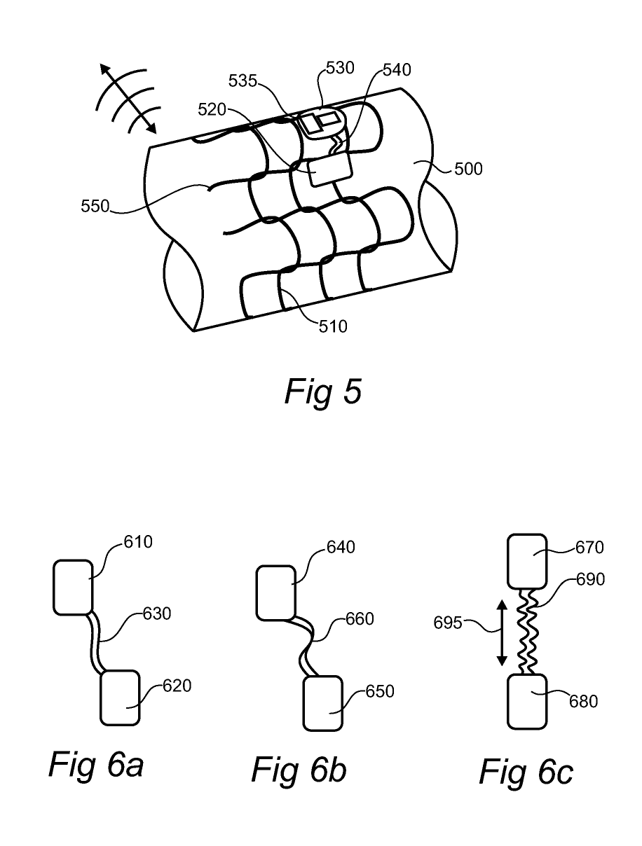

FIG. 5 shows a close up of a system for monitoring the patency of a vascular graft 500 provided in accordance with the present disclosure. The system includes a compliant scaffold 510 formed from one or more wires arranged into a series of compliant loops. The compliant loops may be formed by braiding, weaving, or knitting the one or more wires into a tubular structure. In general, the compliant scaffold 510 may be formed so as to provide a radial mechanical compliance similar to that of an internal thoracic artery when provided in combination with a vascular graft 500. The system further includes one or more sensory modules 530 generally affixed or embedded into the compliant scaffold 510. The system further includes a communication module 520. The communication module 520 may be electrically connected to the one or more sensory modules 530 via one or more links 540. The communication module 520 may be electrically connected to an antenna 550 which may be integrated into the compliant scaffold 510 or alternatively interwoven into the scaffold 510. A power source may also be provided, similarly as described above.

The sensory module 530 and the communication module 520 may be attached to the scaffold 510 in such a way as to minimize influencing the mechanical compliance of the scaffold 510. This may generally be achieved by attaching the sensory module 530 and/or communication module 520 to a wire of the scaffold 510, generally away from any interconnections or joints with other wires of the scaffold 510, e.g., at attachment point 535. The sensory module 530 and/or communication module 520 may be attached to the scaffold 510 using adhesives, melt-bonding, welding, soldering, brazing, mechanically interlocking arrangements, and the like. In general, it may be important that the bonding process is completed without forming any jagged edges or features on the structure of either the modules 520, 530 or the scaffold 510. In addition, if an additive adhesive process may be used to bond the structures, it may be necessary that any biocompatible or bioadhesive coating process that is applied to the resulting system as a whole can still be applied to the bond regions.

The sensory module 530 and/or the communication module 520 may be smaller than the size of a loop of the scaffold 510 so as to minimally influence the mechanical compliance, openness, and profile of the scaffold 510. In order to achieve this goal the modules 520, 530 may be formed from single silicon application specific integrated circuits. It may also be possible to achieve such levels of miniaturization by utilizing wire-bonded or flip chip techniques to bond separate dies to high density interconnect flexible circuits. The one or more links 540 may be formed using HDI flex circuits. In such aspects, the links 540 may further include passive and active components distributed along the link 540 so as to minimally affect the flexibility of the link 540. Alternatively, links 540 may be formed using the approaches outlined above, or similarly to those described below with respect to FIGS. 6a-6c.

The sensory and/or communication modules 530, 520 may also be broken into two or more segments, each segment being sufficiently small so as to fit within a loop of the scaffold 510. The segments may be electrically interconnected using a miniaturized and flexible interconnect. A suitable interconnect may be a stretchable interconnection scheme as outlined above. Alternative interconnects may be micro-wires, HDI flex circuits, or a portion of the compliant scaffold 510.

In aspects, the compliant scaffold 510 may be formed from one or more polymeric sheets or woven from one or more polymeric fibers. Alternatively, the compliant scaffold 510 may be formed from a biocompatible polymer or tissue engineered construct.

Alternatively or additionally, the sensory module 530 may be directly embedded into a tissue engineered graft, tissue or organ.

FIGS. 6a-6c show various flexible links 630, 660, 690 for coupling remotely attached sensory modules 610, 640, 670 and communication modules 620, 650, 680, respectively. Such links 630, 660, 690 may be used in conjunction with any of the sensory modules and communication modules described herein, and/or for coupling any of the other components described herein.

FIG. 6a shows a flexible link 630 formed from a micro-wire bundle. The micro-wire bundle may be formed from a collection of electrically conducting micro-wires, each wire generally having a diameter of less than 1 mm, less than 250 um, less than 100 um, or less than 50 um. The micro-wires may be formed from an electrically conducting metal such as copper. Alternatively, the micro-wires may be formed from a range of alternative electrically conducting metals, electrically conducting polymers, carbon fiber composites, or the like. The micro-wires may be individually isolated with a thin dielectric coating such as polyurethane, nylon, enamel, polytetrafluoroethylene (PTFE), polyester, silicone, or the like. A micro-wire bundle link may be further coated with a biocompatible material so as to minimize foreign body response, improve surface qualities, and the like of the flexible link 630.

FIG. 6b shows a flexible link 660 formed from a flexible substrate. The flexible substrate may be formed from conductor/dielectric laminates with overall thickness of less than 100 um, less than 50 um, less than 25 um, or less than 10 um. The dielectric layers in the laminate may be formed from PTFE, polyimide, polyamide, polyethylene terephthalate, polyethylene naphthalate, or elastomers such as polydimethylsiloxane, polyurethane, and the like. The conducting layers in the laminate may be formed from one or more metals including copper, silver, platinum, gold, nickel titanium, nickel chromium, and the like. Alternatively or in combination, conducting layers maybe formed from carbon (nanofibers, flake, needles, and the like, optionally embedded in a polymeric matrix), conjugated polymers, composites of polymers and metallic filler, and the like. In aspects, the flexible link 660 may be formed from an all-polymer composite. In such aspects, the conducting elements may be formed from highly flexible conducting polymers while the dielectric elements may include suitably soft and biocompatible polymers such as polyurethane, bioadhesives, silicone, biodegradable polymer, and/or a drug eluding composite, growth factors, variable domain antibodies (VHH), anti-migration coatings, and the like.

In aspects, the flexible link 660 may be formed from a composite of polymeric materials including polydimethylsiloxane (PDMS) for the dielectric regions and layers and poly(3,4-ethylenedioxythiophene) (PEDOT) for the conducting regions, layers and traces. Adhesion between layers, especially between layers of different polymers (for example between PEDOT and PDMS regions) may be improved by hydrophilization of the PDMS by means of oxygen plasma or the like. Alternatively, different polymer regions may be compatibilized though use of a silane, a titanate, or other suitable compatibilizing agent.

In aspects, at least a portion of the dielectric regions of the flexible link 660 may be formed from a silicone elastomer such as polydimethylsiloxane, viscoelastic gel, collagen, a porous core elastomer, a perfluoropolyether such as described in US Patent Application Publication Nos. 2005/0142315, 2005/0273146, and 2005/0271784 each of which is incorporated herein by reference in its entirety, a silicone-containing polyurethane, a sufficiently soft polyurethane, PFPE-PDMS block copolymers such as described in U.S. Pat. Nos. 3,810,874, 4,094,911, and 4,440,918 each of which is incorporated herein by reference in its entirety, polyisoprene, polybutadiene such as described in International Patent Application No. PCT/US2010/46072, which is incorporated herein by reference in its entirety, and/or fluoroolefin-basedfluoroelastomers.

FIG. 6c shows a flexible link 690 formed from a stretchable substrate. Such flexible substrates may be formed from composites of semiconducting technologies and elastomeric materials. The specific fabrication method for such circuitry may depend on the specific circuit classes desired to incorporate into the device, and the specific characteristics of the circuitry, including those of the discrete operative devices, the interconnects, etc., include, but are not limited to, those disclosed in the following references each of which is incorporated herein by reference in its entirety: U.S. Pat. No. 7,557,367; Ko et al., “A hemispherical electronic eye camera based on compressible silicon optoelectronics,” Nature (2008); D. -H. Kim, W. M. Choi, J.-H. Ahn, H.-S. Kim, J. Song, Y. Huang, Z. Liu, C. Lu, C G. Koh and J. A. Rogers, “Complementary Metal Oxide Silicon Integrated Circuits Incorporating Monolithically Integrated Stretchable Wavy Interconnects,” Applied Physics Letters 93, 044102 (2008) D.-H.Kim, J.-H.Ahn, W.-M.Choi, H.-S.Kim, T. -H. Kim, J. Song, Y. Y. Huang, L. Zhuangjian, L. Chun and J. A. Rogers, “Stretchable and Foldable Silicon Integrated Circuits,” Science 320, 507-511 (2008); R. Dinyari et al, K. Huang, S. B. Rim, P. B. Catrysse and P. Peumans, “Curved silicon focal plane arrays,” Appl. Phys. Lett. (2008). The following references also provide fabrication methods for such stretchable circuitry and describe the specific characteristics of the circuitry, including those of the discrete operative devices, the interconnects, etc., each of which is incorporated herein by reference in its entirety: US Patent Application Publication Nos. 2006/0286488, 2009/0199960, 2007/0032089, 2008/0157235, and 2008/0108171; U.S. Pat. Nos. 7,195,733 and 7,521,292; and U.S. patent application Ser. Nos. 11/145,574, entitled “Methods and Devices for Fabricating and Assembling Printable Semiconductor Elements, filed Jun. 2, 2005, 11/675,659, entitled “Devices and Methods for Pattern Generation by Ink Lithography,” filed Feb. 16, 2007, and 12/398,811, entitled “Stretchable and Foldable Electronic Devices,” filed on Mar. 5, 2009.

Integration of circuits onto a flexible and/or elastomeric substrate via transfer printing of partially or wholly processed single-crystal silicon devices can be achieved using methods described in the aforementioned references or also methods in M. A. Meitl, Z. -T. Zhu, V. Kumar, K. J. Lee, X. Feng, Y. Y. Huang, I. Adesida, R. G. Nuzzo and J. A. Rogers, “Transfer Printing by Kinetic Control of Adhesion to an Elastomeric Stamp,” Nature Materials 5, 33-38 (2006), which is incorporated herein by reference in its entirety.

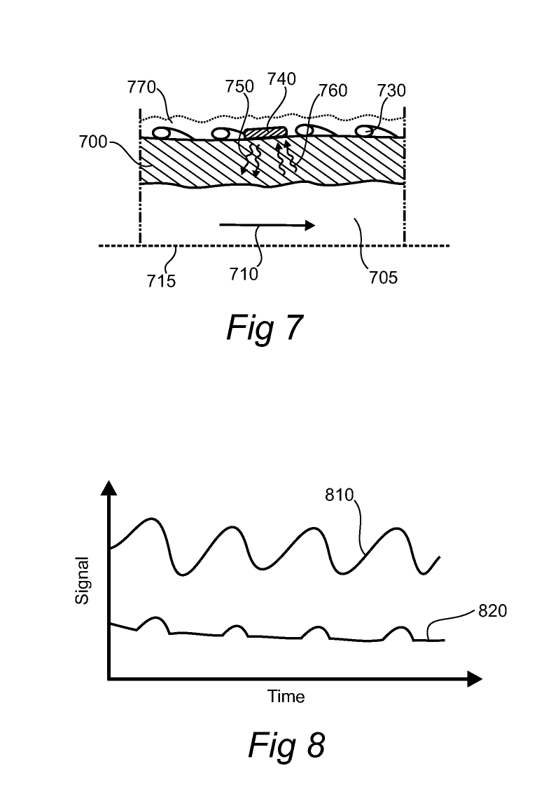

FIG. 7 shows an electro-optic sensory module 740 positioned within a vascular graft 700. In particular, FIG. 7 shows a cross section of vascular graft 700, including lumen 705, which is adapted to accommodate blood flow there through (as indicated by arrow 710) and defines a lumen axis 715 oriented along the lumen 705 of the vascular graft 700. The vascular graft 700 has a wall onto which is affixed a compliant scaffold 730. The compliant scaffold 730 includes a sensory module 740 integrated or otherwise affixed to compliant scaffold 730 in accordance with any of the aspects or configurations described above. The sensory module 740 includes a light source oriented so as to project light towards the lumen 705 of the vascular graft 700, as indicated by arrows 750, and a light detector or photodiode oriented so as to accept light from the lumen 705 of the vascular graft 700, as indicated by arrows 760. Optionally, a complimentary sensory module (not shown) may be located on the opposite side of the vascular graft 700 for similar purposes. The scaffold 700 and sensory modules 740 may become overgrown with tissue overgrowth 770 after implantation into a body. The sensory modules 740 and other components of the system may be configured so as not to be significantly affected by this tissue overgrowth 770. The sensory module 740 is provided in electrical communication with a communication module, similarly as described herein with respect to any of the other aspects. The communication module may be provided so as to communicate with and optionally receive power from an external entity to the body.

In aspects, the communication module may be powered by an external signal. Once powered, the communication module may be configured to activate one or more of the sensory modules 740, collect data regarding the state of the vascular graft 700, surrounding tissue and/or adjacent tissue, communicate the resulting information to an external entity, and then power back down.

In aspects, the sensory module 740 may be configured to monitor analyte concentration in the wall of the vascular graft 700. In aspects, the sensory module 740 may be configured to monitor light absorbed collectively by the wall of the vascular graft 700 and the blood traveling therethrough (or the same with respect to adjacent and/or surrounding tissue). In such aspects, the source light wavelength may be selected so as to control the depth of penetration into the lumen 705 of the vascular graft 700. In aspects, the peak emitted wavelength of the source light may be selected in the range of 425-475 nm while in other aspects or in combination, a second peak emitted wavelength may be selected in the range of 575-675 nm. Incident light onto the photodiode may be further filtered using polymeric coatings, thin film coatings, cross polarized films, combinations thereof, and/or other techniques known in the art.

In aspects, the photodiode may be configured to accept light from a source located outside of the body.

FIG. 8 shows exemplary waveforms 810, 820 obtained from an electro-optical sensory module related to blood flow through the vascular graft, e.g., as in the system shown in FIG. 7. In particular, FIG. 8 demonstrates temporal waveforms of the received signal on a photodiode as polled by an external reader over time. In the case of normal or acceptable blood flow through the vascular graft, as indicated by waveform 810, the temporal waveform generally undulates with the blood flow velocity around a first DC offset. In the case of partially or greatly obstructed blood flow though the venous graft, as indicated by waveform 820, the temporal waveform will change. In general, the waveform 820 may become steadier with less flow related undulation and the DC offset may shift. Monitoring of the waveform in the weeks to months following implantation of the vascular graft can thus be used to provide a clear and early warning to an impending obstruction.

In aspects, an array of sensory modules in accordance with the present disclosure, may be configured to collectively determine flow of a fluid through near anatomical structures (i.e., adjacent and surrounding tissues), a lumen, etc. by assessing signal peaks between each pulsation of the waveforms associated with the flow signal (as determined by each of the sensory modules in the array). The system may be configured to compensate for ambient conditions, motion artifacts, etc.

In aspects, the system may be configured to detect myocardial infarction of a subject into which it is implanted. In this aspect, the system may be configured to monitor for changes in the local flow rates, characteristics of the flow waveforms, or the like. Such monitoring may be used to define a region of normal waveforms and abnormal waveforms. In the case of detection of an abnormal waveform, the system may send an alert, notify a defibrillator (e.g. an implanted defibrillator, etc.), apply a local stimulation, or the like.

In aspects, the sensory module 740 and/or communication module may further be configured to determine movement artifacts, e.g., by providing an accelerometer. Readings from the movement artifact sensor may be used to separate movement artifact related disturbances from the blood flow related undulations in the sensory waveform. Thus, sensor fusion of the signals may be used to provide a more accurate and/or reliable indication of blood flow through the vascular graft 700.

Algorithms may also be provided to facilitate the determination of the patency of the vascular graft 700 from the information gleaned from the sensory signals as outlined above.

Monitoring of the waveform may also be utilized in determining pharmacological dosage levels for medication that a patient may take following surgery.

In addition, the waveform or variations thereof may be suitable for obtaining low cost yet effective diagnostic information regarding the overall health of a patient's cardiovascular system following implantation of the vascular graft 700.

In aspects, a plurality of light sources and/or photodiodes may be provided as associated with a range of sensory modules 740 and/or communication modules. In such aspects, light from various sources may be pulsed in and out of phase and generally be accepted by a range of photodiodes in the system. Such an array may be used to provide a photo-absorption map of the vascular graft 700 to further enhance the accuracy, precision, and/or reliability of the system to determine patency of the graft 700.

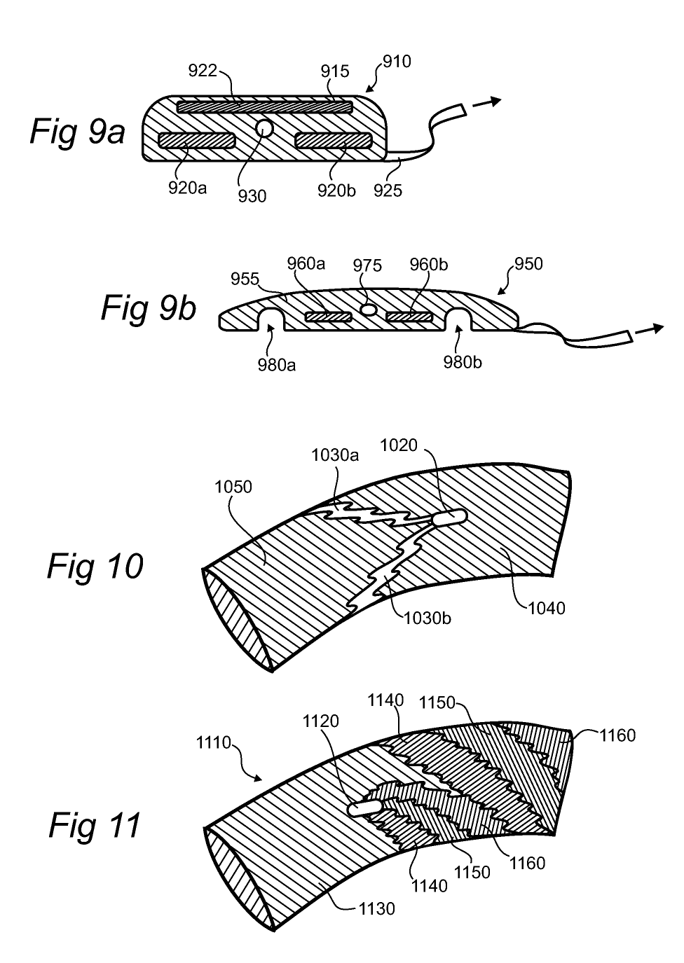

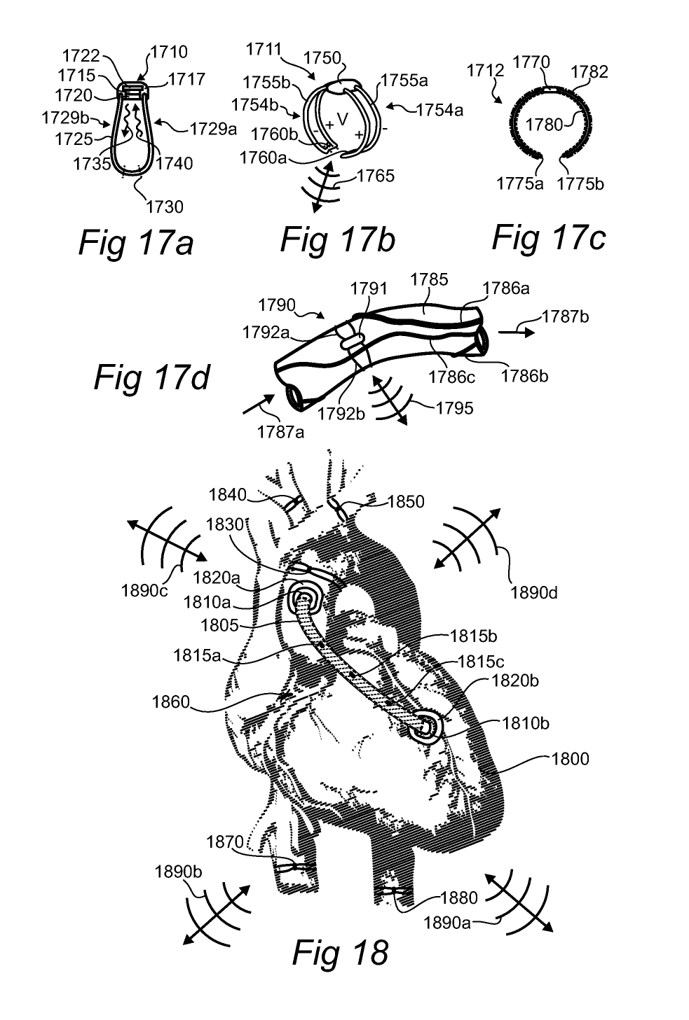

In aspects, and particularly with respect to power saving applications, the sensory data may be processed by an external entity so as to minimize power consumption of the system components located inside the body. In general, power consumption of components within the body is of utmost importance and every effort may be made to minimize on time, power consumption, and the like for any system component within the body. In aspects, a power source may be provided within the body (see, e.g., power supply 122 (FIG. 1), power supply 222 (FIG. 2), power supply 922 (FIG. 9a), and power supply 1722 (FIG. 17a)). In such aspects, moderating power consumption may be especially important so as to minimize the drain on the power source. In such aspects, ultra-low power microelectronic design practices may be strictly utilized to improve performance and battery life.

FIGS. 9a and 9b show sensory modules 910, 950 provided in accordance with the present disclosure and configured for attachment to a compliant scaffold, e.g., any of the complaint scaffolds described herein or any other suitable compliant scaffold.

FIG. 9a shows the sensory module 910, which is configured for attachment to a compliant scaffold via an eyelet 930. The sensory module 910 may include one or more sensor components 920a, 920b configured according to any of the aspects described above for sensing one or more characteristics of a graft, construct, adjacent tissue, and/or surrounding tissue, and a power supply 922 for powering the one or more sensory components 920a, 920b. The sensory module 910 may further includes a flexible link 925 for coupling the sensory module 910 to a communication module that may be configured similarly to the sensory module 910 for similarly attaching the communication module to a compliant scaffold. The eyelet 930 may be a micro-bore through the sensory module 910 suitable for threading a wire of a compliant scaffold therethrough. In aspects, a wire of the scaffold may be conveniently threaded through the eyelet 930 of the sensory module 910 during the fabrication process. As such, using this configuration, a fully functional compliant scaffold with integrated sensory modules and communication modules may be built rapidly in a simple braiding or weaving process. Furthermore, once integrated with the eyelets 930 as outlined above, the integrity of the compliant scaffold with sensory modules 910 may be significantly improved versus other methods of bonding. In addition, eyelets 930 may provide a suitable method for fabricating a low profile, compliant scaffold without jagged edges or surfaces that could potentially lead to wear and damage after implantation into a body.

The power supply 922 incorporated into the sensory module 910 may be configured to only maintain sufficient power such that operation can be maintained while being monitored by an external reader. In aspects where a longer term operation is necessary, the power supply 922 may be configured to store sufficient energy to last between remote recharge cycles (12 hours, 24 hours, etc.). In either case, the supply recharge interval is sacrificed in order to keep the size of the power supply 922 sufficiently small such that it may be mounted on or incorporated into the graft, scaffold, or other implant, while minimizing forces acting thereon.

FIG. 9b shows the sensory module 950 that, similar to sensory module 910, is configured for attachment to a compliant scaffold. More specifically, an eyelet 975 and one or more slots 980a, 980b may be provided for attaching the sensory module 950 to a compliant scaffold. The eyelet 930 may be used as described above with respect to FIG. 9a. The slots 980a, 980b may be used to further reinforce orientation of the sensory module 950 after integration with the scaffold. The slots 980a, 980b may also provide enhanced stability after fabrication and prior to integration of the scaffold with a vascular graft. The sensory module 950 may otherwise be configured similarly to sensory module 910 (FIG. 9a) and/or a communication module similar to sensory module 950 for attachment to the complaint scaffold may also be provided.

In aspects, the sensory modules 910, 950 and/or the communication modules may further include a modeled or micro-structured surface 915, 955. Such a surface 915, 955 may be used to improve uptake of bioadhesive, or ingrowth of surrounding tissue into the modules after implantation.

FIG. 10 shows an antenna 1030a, 1030b woven into a compliant scaffold 1010. In aspects, the scaffold 1010 may be formed from two or more micro-wires, the micro-wires being optionally coated with a thin dielectric coating. In general, the compliant scaffold 1010 may be woven or so constructed as to have an antenna region 1050 and a non-antenna region 1040. The antenna region 1050 may be woven such that the electrical interconnect between wires in the antenna region 1050 may be sufficient so as to provide RF wave propagation along the antenna region 1050 of the scaffold 1010 during use. The wires used in the antenna region 1050 may be electrically conducting and may or may not be coated with a thin dielectric layer (such as a polymeric layer, an oxide coating, etc.). The antenna region 1050 of the compliant scaffold 1010 may be electrically connected with the communication module 1020.

The above configuration may provide a particularly compact compliant scaffold 1010 with a sufficiently large antenna 1030a, 1030b so as to effectively communicate with an outside entity. Furthermore, the effective interweaving of the antenna region 1050 with the non-antenna region 1040 of the scaffold 1010 may facilitate integrating an efficient antenna 1030a, 1030b into the scaffold 1010 without significantly impacting the compliance of the scaffold 1010.

FIG. 11 shows a strategically woven compliant scaffold 1110 with multiple electrically addressable regions 1140, 1150, 1160. In aspects, different electrically accessible regions 1140, 1150, 1160 of the compliant scaffold 1110 may be designed by interweaving multiple electrically isolated wires into the scaffold 1110 during fabrication. As shown in FIG. 11, the communication module 1120 is electrically interconnected with three separate regions, e.g., a first region 1140, a second region 1150, and a third region 1160, of the scaffold 1110. The communication module 1120 may then send signals to or receive signals from the associated regions 1140, 1150, 1160 so as to pole the bioimpedance, or other characteristics of tissues adjacent, surrounding, and/or between the regions 1140, 1150, 1160. Also shown is a null region 1130 designated as a region that is not electrically addressable by the communication module 1120. In an alternative aspect, the null region 1130 may be electrically addressable by the communication module 1120 and used as a reference electrode.

The wires of each region 1140, 1150, 1160 may be electrically isolated from each other as well as internally through use of thin dielectric coatings or oxide layers. A particular segment of the region 1140, 1150, 1160 may be made electrically accessible to the surrounding tissues by local removal of the associated dielectric layer or oxide. Furthermore, a biocompatible conducting coating may be added to these regions 1140, 1150, 1160 to further improve interaction between the region 1140, 1150, 1160 and the surrounding tissues of the vascular graft and body.

FIG. 12 shows another strategically woven compliant scaffold 1210 provided in accordance with the present disclosure and including multiple electrically addressable regions 1240a, 1240b, 1240c. In aspects, the first region 1240a, second region 1240b, and third region 1240c may be physically separated by a null region 1220 which is mechanically interconnected with the other regions 1240a, 1240b, 1240c, but electrically isolating. In the aspect shown, the first region 1240a, second region 1240b, and third region 1240c may be organized so as to provide a three electrode cell (although greater or fewer may alternatively be provided) for assessing electrochemical properties of the vascular graft, fluid within the graft, surrounding tissues and/or adjacent tissues.

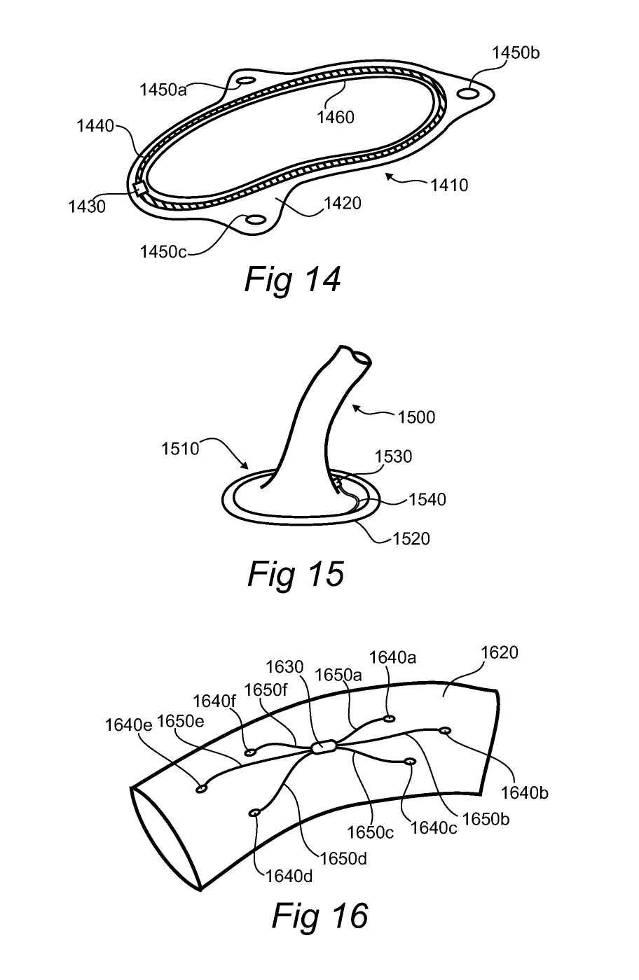

FIG. 13 shows two ringlet sensors 1320a, 1320b provided in accordance with the present disclosure and located at different ends of a vascular graft 1310 for monitoring patency of the vascular graft 1310. The ringlet sensors 1320a, 1320b may be arranged so as to monitor flow of blood into, out of, and/or through the vascular graft 1310. The ringlet sensors 1320a, 1320b need not be provided in physical contact with the vascular graft 1310. The ringlet sensors 1320a, 1320b may be attached to the heart 1330 or arteries 1340 during a CABG procedure. The ringlet sensors 1320a, 1320b each generally include a communication module similar to any of those described above, an antenna similar to any of those described above, and/or an electromagnetic field generating electrode set or coil, e.g., similar to coil 1440 (FIG. 14). The communication module may drive the EM electrode set or coil to form a magnetic field within the vascular graft 1310. Flow of blood through the generated field will create a current within the electrode set or coil 1440 (FIG. 14) that can be monitored by the communication module.

In alternative aspects, an EM field may be generated through the EM electrode set or coil 1440 (FIG. 14) by an external EM source. During excitation, the communication module may monitor both the EM excitation of the electrode set or coil 1440 (FIG. 14) as well as the current formed by fluid flow through the coil 1440 (FIG. 14). The communication module may store the related measurements in memory and communicate this information to an external entity for further analysis. Thus, the associated measurement may be made using a minimal of power on the communication module.

FIG. 14 shows a ringlet sensor 1410 for monitoring patency of a vascular graft. As shown, the ringlet sensor 1410 includes a housing 1460 that retains a coil 1440 that may dually function as a field generator for assessing flow through a vascular graft, as discussed above, and an antenna for communication to an external entity outside the body. The ringlet sensor 1410 may also include a communication module 1430 that may be provided in electrical communication with the coil 1440. The communication module 1430 may include memory, a processor, power management circuitry, RF circuitry, and the like to suitably monitor flow through the associated vascular graft as well as communicate with an outside entity. Also shown, an optional array of attachment points, or apertures, 1450a, 1450b, 1450c may be used to attach the ringlet sensor 1410 to the heart (or other organ or tissue) around the associated vascular graft.

In general, the ringlet sensor 1410 may be sufficiently soft and flexible so as to not impede movement of the heart or vascular graft. In addition, the ringlet sensor 1410 may be suitably soft and smooth so as to minimize abrasive damage to adjacent surfaces after implantation of the ringlet sensor 1410 and vascular graft.

In alternative aspects, the ringlet sensor 1410 may be integrated into the end of the vascular graft as an artificial anastomotic connector. In such aspects, the ringlet sensor 1410 may further include tissue adhesives coating attachment points 1450a, 1450b, 1450c and the like to provide a convenient mechanism for interconnecting a vascular graft to tissue, e.g., heart and/or artery tissue. In this case, the ringlet sensor 1410 may provide a fillet 1420 for enhancing the contact area of the interconnect, thereby strengthening the interconnection between the graft and the heart, artery, or other tissue structure.

The ringlet sensor 1410 may be formed from an all-polymer interconnect or a stretchable semiconducting element as outlined above.

FIG. 15 shows another ringlet sensor 1510 for monitoring patency of a vascular graft 1500. As shown, ringlet sensor 1510 includes a further sensory module 1530 electrically connected with a ringlet 1520. The ringlet 1520 may include the antenna, EM coil or electrodes, and the communication module as described above with respect to ringlet sensor 1410 (FIG. 14).The sensory module 1530 may be connected to the communication module of the ringlet 1520 using a flexible link 1540. The sensory module 1530 may be similar to those described above. In addition, the sensory module 1530 may be affixed to the vascular graft 1500 using a bioadhesive, a suture, a staple or via any other associated method. The sensory module 1530 may be adapted so as to obtain tissue health, blood flow, or related information regarding the graft 1500, surrounding tissue and/or adjacent tissue to further enhance the accuracy and/or reliability of the ringlet sensor 1510 in assessing the patency of the vascular graft 1500.

FIG. 16 shows an electrically shielded system provided in accordance with the present disclosure for monitoring a vascular graft. The system includes an electrically conductive scaffold 1620 arranged so as to provide a Faraday cage around a vascular graft. The system further includes a communication module 1630 and a plurality of sensory modules 1640a-f arranged within the volume formed by the electrically conductive compliant scaffold 1620. The communication module 1630 and sensory modules 1640a-f may be provided in electrical communication using one or more flexible links 1650a-f, respectively, similarly as described above.

In aspects, the sensory modules 1640a-f may be configured so as to monitor the bioimpedance of a vascular graft and blood flow there through after implantation into a body, as well as to monitor surrounding and/or adjacent tissue. Collectively, sensory data obtained by poling the network of sensory modules 1640a-f can be used to formulate an impedance map of a vascular graft and the bloodflow therethrough after implantation into the body. In such aspects, the electrically conducting compliant scaffold 1620 may provide a simplified and effective system for isolating the impedance sensory network from the surrounding tissues. Thus, implementation of a conducting compliant scaffold 1620 in combination with the network of sensory modules 1640a-f may be suitable for creating precise and/or accurate assessment of a vascular graft after implantation into a body.

FIGS. 17a-d show various clip-like systems 1710, 1711, 1712 for monitoring a site within a body in accordance with the present disclosure. FIG. 17a shows a side view of a clip-like system 1710 for attachment to a tubular structure (e.g. a tubule, a graft, a vessel, a blood vessel, an artery, a carotid artery, a renal artery, a vein, a venule, a nerve, a nerve bundle, a plexus, a renal plexus, a urethra, a ureter, a lymphatic vessel, etc.). The clip-like system 1710 includes a housing 1717, the housing 1717 optionally including one or more sensory modules 1720, a communication module 1715, and a power supply 1722, provided in accordance with the present disclosure (and configured similarly to any of those discussed above). The clip-like system 1710 includes one or more legs 1725 mechanically connected to the communication module 1715. The legs 1725 may be configured in loop formations (as shown) as individual supports (e.g. curved wires, etc.) so as to enwrap, interface with, and/or attach the system 1710 to an adjacent tubular structure. One or more of the legs 1725 may be formed from a super elastic material (e.g., a shape memory alloy, a nickel titanium alloy, etc.)

so as to provide sufficiently reversible deformation so as to fit around the tubular structure during attachment but retain a snug fit between the clip-like system 1710 and the tubular structure after attachment. One or more of the legs 1725 may be trained to retain a first shape (e.g. a substantially closed shape, retaining ring like shape, etc.).

In one non-limiting example, one or more legs 1725 may be formed such that at a temperature substantially below body temperature (e.g. less than 0 C, less than 20 C, less than 30 C, etc.) the legs 1725 may be substantially plastically deformable so as to be easily bent around a tubular structure. Upon warming (e.g. provided via heat transfer to the adjacent anatomy, tubular structures, via thermal transfer from a placement tool, etc.) the legs 1725 may be configured to wrap around the adjacent tubular structure, so as to intimately interface therewith.

One or more of the legs 1725 may be configured with electrically interfaceable regions. In one non-limiting example, the legs 1725 may include insulating regions 1729a, 1729b (e.g., including an electrically insulating material so as to conductively isolate the leg 1725 from adjacent anatomy in the vicinity of the region), and/or conducting regions 1730 with electrically conductive surfaces (e.g., including an electrically conducting material so as to conductively interface the leg 1725 with adjacent anatomy during use). Such regions 1729a, 1729b, 1730 may be advantageous for selectively interfacing with the adjacent tubule during operation, stimulating local tissues, monitoring one or more evoked potential local at sites along the tubule, monitoring electrical impedance between regions of the tubule, monitoring neuronal activity, monitoring electromyographic signals, etc.

One or more of the sensory modules 1720 may include one or more sensors and/or stimulators each in accordance with the present disclosure. In one non-limiting example, one or more of the sensory modules 1720 may include a photosource and/or photodetector. The photosource may emit radiation 1735 towards an adjacent anatomical structure (e.g. a tubule), and the photodetector may monitor radiation 1740 emitted, reflected or transferred thereto via the surrounding and/or adjacent anatomical structures during use. Such a sensory module 1720 may be advantageous for assessing the adjacent and/or surrounding anatomical structures even in the event that tissue growth around the sensory module 1720 may substantially isolate the sensory module 1720 from the adjacent and/or surrounding anatomical structure during use, similarly as described above.

In one non-limiting example, one or more of the legs 1725 may be connected in electrical communication with the communication module 1715 so as to form at least a portion of an antenna. The legs 1725 may be arranged with the appropriate dimensions so as to at least somewhat efficiently behave as an antenna at the intended wavelength of communication. In one non-limiting example, a pair of legs 1725 may form a dipole antenna structure.

In one non-limiting example, the legs 1725 may be configured to provide multiple capabilities including physically interfacing with an adjacent and/or surrounding anatomical structure or tissue, interfacing with the local anatomical structure, and/or acting as an antenna for communication between the communication module 1715 and an associated reader.

FIG. 17b shows clip-like system 1711 for monitoring adjacent and/or surrounding anatomical structure or tissue within a body. The clip-like system 1711 may include a housing 1750 and one or more legs 1754a, 1754b in accordance with the present disclosure. The housing 1750 may include one or more sensory modules and/or communication modules (similarly as in FIG. 17a or any other aspects of the present disclosure). The legs 1754a, 1754b may include one or more conducting regions 1755a, 1755b and/or one or more insulating regions 1760a, 1760b. The conducting regions 1755a, 1755b on the same leg 1754a, 1754b may be isolated from each other by one or more of the insulating regions 1760a, 1760b. The legs 1754a, 1754b may be electrically interfaced with the sensory module (not explicitly shown) such that the sensory module may interface with the adjacent and/or surrounding anatomical structure or tissue during use. In one non-limiting example, similarly as mentioned above, the sensory module may function as an electrode to stimulate the anatomical structure or tissue by applying a voltage, current and/or charge between one or more of the conductive regions 1760a, 1760b and/or monitor an electropotential, charge accumulation, etc. there between during operation.

One or more of the legs 1754a, 1754b may be connected in electrical communication with the communication module (not explicitly shown) so as to form as least a portion of an antenna. Thus, the legs 1754a, 1754b may be used to assist with communicating a signal 1765 between the communication module and an associated reader.

FIG. 17c shows clip-like system 1712 for monitoring an adjacent anatomical site within a body in accordance with the present disclosure. The clip-like system 1712 may include a housing 1770 and one or more legs 1775a, 1775b both in accordance with the present disclosure. The clip-like system 1712 may include an interfacing material 1780, 1782 configured to provide some function during use. The interfacing material 1780 may be arranged so as to interface between one or more of the legs 1775a, 1775b and surrounding/adjacent tissue during use. The interfacing material 1780, 1782 may include a drug delivery layer, a scaffolding material, a stress relief mesh, a degradable material, an electrically isolating material, combinations thereof, or the like. In one non-limiting example, the interfacing material 1780 may include a degradable material (e.g. a biodegradable material, bioresorbable material, a water soluble material, etc.). Some non-limiting examples of biodegradable materials include polyesters, polyorthoesters, polyanhydrides, polycaprolactone (PCL), polylactide (PLA), polyglycolide (PGA), bioglass®, silk, metallic glasses (Ca-based glasses, etc.), organic electronic materials, combinations thereof, or the like. Such interfacing material 1780, 1782 may be adhesively attached, solvent cast, electrospun, and/or powder coated, onto one or more aspects of the clip-like system 1712.

In one non-limiting example, the clip-like system 1712 may include one or more biodegradable interfacing materials 1780, 1782. The biodegradable interfacing material 1780, 1782 may facilitate firm interaction with the adjacent anatomical structure or tissue during placement. Over time the biodegradable interfacing material 1780, 1782 may degrade, thus altering the physical properties of the clip-like system 1712, altering the interfacing properties, etc. In one non-limiting example, the biodegradable interfacing material 1780, 1782 may provide an initial structure support for one or more electrical interfacing aspects (e.g., circuitry interconnected with one or more of the sensory modules and/or communication modules), upon degradation of the biodegradable material 1780, 1782 the electrical interfacing aspects may be more intimately interfaced with adjacent/surrounding tissues, thus altering the interfacial impedance, changing the available signals that may be read therefrom, signifying the degree of completion of the degradation process, etc.

FIG. 17d shows a clip-like system 1790 for monitoring an adjacent anatomical structure or tissue 1785 (e.g., a vessel, a blood vessel, an artery, a vein, a vascular graft, etc.) in accordance with the present disclosure. The clip-like system 1790 includes a housing 1791 configured to house one or more sensory modules and/or communication modules each in accordance with the present disclosure. One or more of the sensory modules may be configured to monitor one or more aspects of fluid flow 1787a, 1787b through the adjacent anatomical structure or tissue 1785. The fluid flow 1787a, 1787b may be monitored so as to assess stenosis within the adjacent anatomical structure 1785, so as to assess perfusion of fluids to the tissues within the adjacent anatomical structure 1785, etc. The clip-like system 1790 may include one or more legs 1792a, 1792b in accordance with the present disclosure. The clip-like system 1790 may be arranged so as to monitor one or more neurological structures 1786a-c during use (e.g., a nerve, a nerve bundle, a nerve plexus, etc.).

The communication module may be configured to communicate one or more signals 1795 with an associated reader, similarly as described above. In one non-limiting example, the communication module may be connected with one or more of the legs 1792a, 1792b. Thus one or more of the legs 1792a, 1792b may be configured to perform as at least part of an antenna function.

FIG. 18 shows a multi-component system for monitoring one or more functional aspects of tissue such as an organ 1800 (e.g., a heart, a liver, a kidney, an arterial branch, a lung, etc.) in accordance with the present disclosure. The multi-component system may include one or more grafts 1805, optionally including a flexible scaffold in accordance with the present disclosure. The graft 1805 may include one or more sensory modules 1815a-c in accordance with the present disclosure. Located along with one or more of the sensory modules 1815a-c, the multi-component system may include one or more communication modules (such as any of those described above) configured to communicate between one or more of the sensory modules 1815a-c and/or an associated reader (not explicitly shown). The graft 1805 may be attached to the organ 1800 at a first end 1810a and a second end 1810b (although a plurality of ends may be desirable depending on a particular purpose). The multi-component system may include one or more ringlet sensors 1820a, 1820b in accordance with the present disclosure.

One or more of the sensory modules 1815a-c may monitor fluid flow through the graft 1805 at points along the length thereof. Signal variations, waveform variations, and/or temporal delays in the signal analysis may be used by the system, an associated reader, and/or an external analysis center (e.g., a cloud based computational network, a tablet computer, etc.) to generate one or more flow related metrics from the combination of signals from each of the sensory modules 1815a-c.

In one non-limiting example, the sensory modules 1815a-c may be configured to monitor blood flow through the graft 1805. The pulsatile nature of the blood flow signal (e.g., as illustrated by the waveforms shown in FIG. 8) leads to rising and falling edges. Such signals may be monitored at each of the sensory modules 1815a-c during use. The temporal delay between rising and or falling edges at each sensory module 1815a-c along the graft 1805 may be related to the flow rate there through.

The multi-component system may include one or more clip-like systems 1830, 1840, 1850, 1860, 1870, 1880 for monitoring one or more functions at sites on, near, or related to the organ 1800. In one non-limiting example, the clip-like systems 1830, 1840, 1850, 1860, 1870, 1880 may be configured to monitor local perfusion and/or fluid flow in the nearby anatomical structures to generate associated sensory signals. The sensory signals may be collectively assessed in order to elucidate the global function of the organ 1800 being monitored. In the case that the organ may be a heart, the clip-like systems 1830, 1840, 1850, 1860, 1870, 1880 may monitor blood flow towards and/or away from the heart during use. Such information may be used to assess cardiac output, overall heart health, help locate disease sites, or diagnose disease states during use, etc.

One or more of the communication modules included in the associated clip-like systems 1830, 1840, 1850, 1860, 1870, 1880, ringlet sensors 1810a, 1810b, graft 1805, etc. may communication via signals 1890a-d with an associated reader, amongst associated communication modules, etc. during use, similarly as described above. In one non-limiting example, the monitored signals may be communicated by the associated communication modules to a centralized computational client, whereby timing delays, signal content, etc. may be assessed from the collective sensory systems within the body. Such an assessment may be used to determine more globalized function of the organ 1800 or the body.

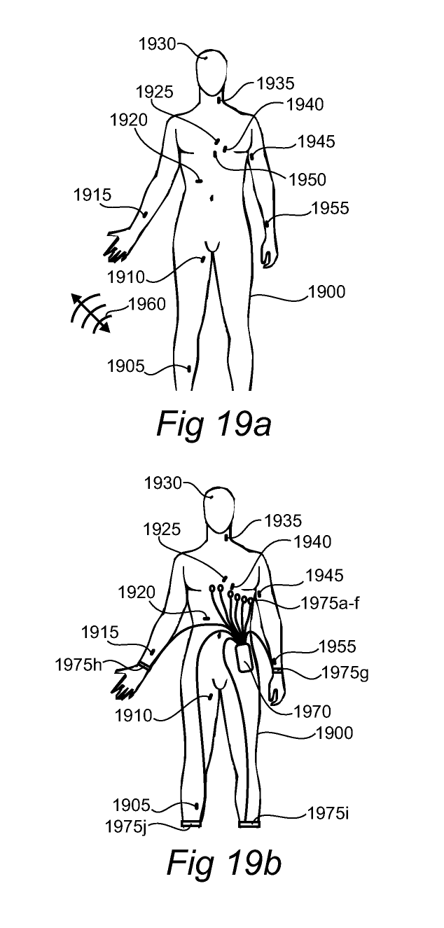

FIGS. 19a and 19b show non-limiting examples of multi-component systems for monitoring one or more bodily functions of a subject (e.g. cardiovascular function, urological function, brain blood perfusion, etc.) in accordance with the present disclosure. FIG. 19a shows a plurality of sensory systems 1905, 1910, 1915, 1920, 1925, 1930, 1935, 1940, 1945, 1950, 1955 situated throughout a body 1900 during use. Each sensory system 1905, 1910, 1915, 1920, 1925, 1930, 1935, 1940, 1945, 1950, 1955 may include one or more sensory modules and/or one or more communication modules each in accordance with the present disclosure. Each sensory system 1905, 1910, 1915, 1920, 1925, 1930, 1935, 1940, 1945, 1950, 1955 may monitor local physiological function and generate associated signals 1960 to be delivered from the body 1900 by associated communication modules in accordance with the present disclosure. Collectively, information provided by one or more of the associated signals 1960 may be used to determine performance, health, tissue healing, patency of one or more vessels, of a subject for purposes of health and/or disease monitoring, diagnostic function, treatment planning, treatment monitoring, prognostication on the state of a diseased site, a stroke warning system, etc.

Such a system may be used to determine overall performance of a bodily function (e.g., cardiovascular performance, post-operative healing, etc.), blood flow through regions of the body (e.g., blood flow through the brain, blood perfusion within the brain), fluid flow throughout the body (e.g., urine flow in the bladder, etc.), blood flow in the extremities (e.g., arms, legs, etc.), fluid flow through one or more organs (e.g., a kidney, a liver, a heart, a lung, a sinus, a lymphatic duct, etc.).

One or more of the sensory systems 1905, 1910, 1915, 1920, 1925, 1930, 1935, 1940, 1945, 1950, 1955 may be strategically placed during a surgical and/or interventional procedure. The selection of the placement site(s) may be determined based upon the need of the surgical indication in question.

FIG. 19b shows a multi-component system for determining cardiovascular performance in a subject. Similarly to the system of FIG. 19a, the multi-component system includes a plurality of sensory systems 1905, 1910, 1915, 1920, 1925, 1930, 1935, 1940, 1945, 1950, 1955 in accordance with the present disclosure, each sensory system 1905, 1910, 1915, 1920, 1925, 1930, 1935, 1940, 1945, 1950, 1955 placed so as to assess one or more aspects of the cardiovascular performance of the subject. The multi-component system may further include one or more extracorporeal sensors, configured to further assess cardiovascular function. Shown in FIG. 19b, the multi-component system includes a twelve lead electrocardiography system 1970 including patient leads 1975a-j attachably configured to interface with the subject 1900 during setup and use and to monitor one or more biosignals of the subject 1900. The extracorporeal sensors 1970 may provide electrocardiographic information (i.e., twelve lead ECG as shown in FIG. 19b) for combination with sensory signals generated by one or more of the sensory systems 1905, 1910, 1915, 1920, 1925, 1930, 1935, 1940, 1945, 1950, 1955 during use. Such a system may provide a simplified system for monitoring cardiovascular performance in a subject 1900 without significantly limiting mobility, while the subject 1900 may be in an uncontrolled environment, etc.

One or more of the extracorporeal sensors 1970 may include a reader for communicating with one or more of the sensory systems 1905, 1910, 1915, 1920, 1925, 1930, 1935, 1940, 1945, 1950, 1955. The collective data may be analyzed amongst one or more of the sensors (e.g., extracorporeal sensors 1970, one or more sensory systems 1905, 1910, 1915, 1920, 1925, 1930, 1935, 1940, 1945, 1950, 1955, etc.). Alternatively, additionally, or in combination, the collective data may be sent to a computational center (e.g., a laptop, a tablet computer, a smartphone, a router, a server, a cloud based network, etc.) for analysis and determination of disease specific information therefrom.

In general the above disclosure can be considered when implementing an aspect as related to a stent instead of as a graft. As the stent may be placed inside a vessel and may be subjected to large expansion stresses during the implantation procedure, some significant differences in design may be considered. In the case of a vascular graft, the compliant scaffold may be made elastic so as to deform and restore its shape in a biomimetic fashion. Alternatively, stents may be formed to take a plastically deformed shape after the expansion process. Attachment points between the stent and the sensory module, communication module, and antenna may be subjected to large stresses and thus careful design maybe necessary to limit device failures in practice. The stent based system may include, incorporated into to one or more of the sensory modules, one or more vibrating sensors, thermal mass flow sensors, pressure gradient based flow sensors, combinations thereof, and the like to assess blood flow there through, local blood turbulence, etc.

In addition, since the sensory module, communication module, power supply, and antenna (optionally provided as the stent itself or a portion thereof), may be provided within the vessel, implementation materials, shapes, and profiles with low thrombogenicity may be more strict than for systems configured for placement on the outside of a vessel.

In aspects, a system in accordance with the present disclosure may be configured to monitor one or more physiological parameters for a prolonged period after implantation of the system in a subject. The system may be configured, optionally in conjunction with an external entity, to monitor one or more physiological parameters, perhaps related to graft patency, heart function, blood flow, etc. for more than 6 months, 12 months, or 24 months after implantation thereof. Such information may be used in combination with an associated database to assess restenosis rates, population segmented restenosis rates, effectiveness of a therapy, etc. amongst an extended patient population. Such information may be useful for assisting a physician with treatment decision making, etc. related to a specific patient within the overall patient population under study.

In aspects, a system in accordance with the present disclosure may be configured as a distributed pacing and/or sensing system. The system may include a stimulation module (incorporated into or separate from the sensory module), the stimulation module further configured for electrically stimulating local tissues. The stimulation module may be configured to provide pacing function to the adjacent tissues, stimulate local tissues for sensing purposes, to provide a timing signal for incorporation amongst other, possibly remotely located systems, and the like. As such, the system maybe configured as a distributed cardiac pacemaker.

In aspects, one or more system components (sensory modules, communication modules, stimulation modules, power supply) may be embedded into a tissue engineered myocardium. The implantation of which, or even addition of which to the heart may be used to enhance heart function and/or provide associated pacing and monitoring functionality without the need for a large separate control unit as seen in traditional pacemakers. The system may include a power source in accordance with the present disclosure.

It will be appreciated that additional advantages and modifications will readily occur to those skilled in the art. Therefore, the invention and its broader aspects are not limited to the specific details and representative aspects shown and described herein. Accordingly, many modifications, equivalents, and improvements may be included without departing from the spirit or scope of the general inventive concept as defined by the appended claims and their equivalents.

1-36. (canceled)

37. A system for monitoring a body, comprising:
a surgical implant configured for implantation into a body;
a sensory module coupled to the surgical implant and configured for implantation into the body in conjunction with the surgical implant, the sensory module configured to monitor characteristics of at least one of the surgical implant, surrounding tissue, and adjacent tissue, the sensory module including at least one light source configured to illuminate at least one of the surgical implant, surrounding tissue, or adjacent tissue, and at least one photodetector configured to receive light from the at least one of the surgical implant, surrounding tissue, or adjacent tissue; and
a communication module coupled to the surgical implant and configured for implantation into the body in conjunction with the surgical implant, the communication module electrically coupled to the sensory module and configured to communicate a signal derived from the characteristics to an external entity.

38. The system according to claim 37, wherein the surgical implant includes a compliant scaffold, and wherein the sensory module and the communication module are affixed to the compliant scaffold.

39. The system according to claim 38, wherein the compliant scaffold is the surgical implant.

40. The system according to claim 38, wherein the surgical implant is a vascular graft and wherein the compliant scaffold is configured for positioning about the vascular graft.

41. The system according to claim 37, wherein the surgical implant is a tissue engineered construct.

42. The system according to claim 38, wherein the surgical implant is a tissue engineered construct, and at least one of the compliant scaffold and the sensory module is at least partially embedded within the tissue engineered construct.

43. The system according to claim 38, wherein the communication module is electrically connected to the compliant scaffold and at least a portion of the compliant scaffold provides an antenna function configured to facilitate communication with the external entity.

44. The system according to claim 37, further comprising a power supply disposed in electrical communication with at least one of the communication module and the sensory module, wherein at least one of the sensory module, the communication module, and the power supply is electrically connected by at least one flexible link.

45. The system according to claim 44, wherein the at least one flexible link is formed from a stretchable interconnect including at least one electrically insulating region and at least one electrically conducting region.

46. A system for monitoring patency of a vascular graft, the system comprising:
a compliant scaffold formed about a vascular graft;
a sensory module affixed to the compliant scaffold and configured to monitor characteristics of at least one of the vascular graft, surrounding tissue, and adjacent tissue;
a communication module affixed to the complaint scaffold and electrically coupled to the sensory module; and
an antenna affixed to the compliant scaffold and electrically coupled to the communication module, wherein the sensory module includes at least one light source directed towards the vascular graft and at least one photodiode and/or photodetector directed towards the vascular graft.

47. The system according to claim 46, wherein the antenna is formed from flexible conducting material configured to conform to a surface of the compliant scaffold.

48. The system according to claim 47, wherein the sensory module is configured to monitor blood flow through the vascular graft.

49. A self-diagnostic system, comprising:
a tissue engineered construct configured for compatibility with body tissue; and
a sensory module at least partially embedded into the tissue engineered construct, the sensory module configured to monitor characteristics of at least one of the tissue engineered construct, surrounding tissue, or adjacent tissue.

50. The self-diagnostic system according to claim 49, wherein the sensory module includes at least one light source configured to illuminate at least one of the tissue engineered construct, surrounding tissue, or adjacent tissue, at least one photodetector configured to receive light from the at least one of the tissue engineered construct, surrounding tissue, or adjacent tissue, and/or at least one electrode configured to electrically interface with at least one of the tissue engineered construct, surrounding tissue, or adjacent tissue.

51. The self-diagnostic system according to claim 49, further comprising a communication module at least partially embedded into the tissue engineered construct and electrically coupled to the sensory module, the communication module configured to communicate a signal derived from said characteristics to an external entity.

52. The self-diagnostic system according to claim 51, wherein at least one of the sensory modules and the communication module is electrically connected by at least one flexible link.

53. The self-diagnostic system according to claim 52, wherein the at least one flexible link is formed from a stretchable interconnect including at least one electrically insulating region and at least one electrically conducting region.

54. The self-diagnostic system according to claim 53, wherein the tissue engineered construct is fabricated so as to mimic a body vessel.

55. The self-diagnostic system according to claim 49, wherein the sensory module is configured to monitor blood flow through the tissue engineered construct.

56. The self-diagnostic system according to claim 49, wherein the tissue engineered construct is fabricated so as to mimic at least a portion of a heart.
